SEQUENCE LISTING (29 sequences)

SEQ ID NO: 1 - DNA, 2694 bp, Homo sapiens
  misc_feature (1)..(2694): Human CtiP
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatag

SEQ ID NO: 2 - protein, 897 aa, Homo sapiens
  MISC_FEATURE (1)..(897): Human CtiP
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSLQNFPHIEVVRKKEERRKLLGHTCKECEIYYADMPAEEREKKLASCSRHRFRYIPPNTPENFWEVGFPSTQTCMERGYIKEDLDPCPRPKRRQPYNAIFSPKGKEQKT

SEQ ID NO: 3 - DNA, 2124 bp, Homo sapiens
  misc_feature (1)..(2124): Human MRE11 isoform 1
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagcagacactggtctggagacttctacccgtagcaggaactcaaagactgctgtgtcagcatctagaaatatgtctattatagatgcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaaga

SEQ ID NO: 4 - protein, 708 aa, Homo sapiens
  MISC_FEATURE (1)..(708): Human MRE11 isoform 1
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRADTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 5 - DNA, 2040 bp, Homo sapiens
  misc_feature (1)..(2040): Human MRE11 isoform 2
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaaga

SEQ ID NO: 6 - protein, 680 aa, Homo sapiens
  MISC_FEATURE (1)..(680): Human MRE11 isoform 2
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 7 - DNA, 2121 bp, Homo sapiens
  misc_feature (1)..(2121): Human MRE11 isoform 3
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagacactggtctggagacttctacccgtagcaggaactcaaagactgctgtgtcagcatctagaaatatgtctattatagatgcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaaga

SEQ ID NO: 8 - protein, 707 aa, Homo sapiens
  MISC_FEATURE (1)..(707): Human MRE11 isoform 3
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRDTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 9 - DNA, 420 bp, Homo sapiens
  misc_feature (1)..(420): Human SRS2
atggccgtagtgttgccggcggttgtggaggagctcctgagcgagatggcggcggcggtgcaggagagcgcgcgaattcctgatgaatatctgttatcgctgaagtttctctttggctcatcagccacccaggccttggacctagttgatcgacagtccatcaccttaatctcatcacccagtggaaggcgtgtttaccaggtccttggaagttccagtaaaacatacacatgtttggcttcttgtcattactgttcatgtcctgcatttgcattctcagtgctacggaagagtgacagcatcctgtgcaagcatctcttggcagtttacctgagtcaggttatgaggacctgtcagcagctaagtgtctctgacaagcagttgactgacatattattgatggagaagaaacaagaagca

SEQ ID NO: 10 - protein, 140 aa, Homo sapiens
  MISC_FEATURE (1)..(140): Human SRS2
MAVVLPAVVEELLSEMAAAVQESARIPDEYLLSLKFLFGSSATQALDLVDRQSITLISSPSGRRVYQVLGSSSKTYTCLASCHYCSCPAFAFSVLRKSDSILCKHLLAVYLSQVMRTCQQLSVSDKQLTDILLMEKKQEA

SEQ ID NO: 11 - DNA, 882 bp, Cricetulus griseus
  CDS (1)..(882): CHO Ercc1 (Excision Repair Cross-Complementation Group 1)
atggaccttgggaaagacgagggaagcctgccgcagcccaccaggaagaagtttgtgatcccactggaagacgaggcccctcctgcaggggccaagcccttattcagatcctcacggaaccccagcaccacggccccctcggtcccagcggcccctcagacgtacgccgagtatgccattgcccagcctccaggaggggctgggcccacagggcccacaggctctgaacctgtgaagggagagaaccccggccagacggtgaaaacgggagcgaaatccaatagcatccttgtgagcccccggcagaggggcaaccctgtgttgaagttcgtgcgcaacgtgccctgggaattcggcgaggtgacccctgactatgtgctgggacagagcacttgcgcccttttcctcagcctccgctaccacaatctccatccagactacatccacgaacggctgcagagcctggggaagagctttgccctgcgtgtgctgttggtccaagtggatgtgaaagatcctcagaaggccctgaaggacctggctaaaatgtgtatcttagcggactgcaccctggtcctggcctggagtgccgaggaagcaggacggtacctggagacctacaaggcatatgagcagaagcccgctgacctcctcatggagaagctggagcagaacttcctgtcccgggccaccgagtgtctgaccaccgtgaagtcagtcaacaaaaccgacagccagaccctcctggctacatttggatcccttgaacagctcttgacggcatcacgggaggacctagccttgtgccccggcctgggcccccagaaggcccgcaggctctttgacgtcctccatgaacccttcctcaaagtgccccgatga

SEQ ID NO: 12 - protein, 293 aa, Cricetulus griseus
MDLGKDEGSLPQPTRKKFVIPLEDEAPPAGAKPLFRSSRNPSTTAPSVPAAPQTYAEYAIAQPPGGAGPTGPTGSEPVKGENPGQTVKTGAKSNSILVSPRQRGNPVLKFVRNVPWEFGEVTPDYVLGQSTCALFLSLRYHNLHPDYIHERLQSLGKSFALRVLLVQVDVKDPQKALKDLAKMCILADCTLVLAWSAEEAGRYLETYKAYEQKPADLLMEKLEQNFLSRATECLTTVKSVNKTDSQTLLATFGSLEQLLTASREDLALCPGLGPQKARRLFDVLHEPFLKVPR

SEQ ID NO: 13 - DNA, 3042 bp, Cricetulus griseus
  CDS (1)..(3042): CHO Ligase 3
atgactttggctttcaagatcctcttcccgagaaacctttgtgcccttggcagaaaagaactgtgcctgttctcagaacagaatcactggcctgtcataagacagttcagccagtggtcggaaacagatctccttcgtgggtgctgcctcctccagagaagaaagcctgtcctatctttccagggaggtcatctaagaccacgtgccacccaccttgttttcttcccagggtcgcatgtgggactctatactggcccctatgagatggcggagcagcggttctgtgtggactatgccaagaggggcacagctggttgcaagaaatgcaaggagaagattttaaagggcgtatgccgcattggcaaagtggtgcccaatcccttctcagagtctgcgggcgatatgaaagagtggtaccatgttaagtgcatatttgagaaactggagcgggctcgggctaccacaaaaaaaattgaagacctcacagagctagaaggctgggaagagctggaagatgacgaaaaggaacagatcagccagcacattgcagacctgtcctctaaggcagctgggacacctaagaagaaaaccgctgtccaggctaaggtgacaaccactggccaggtgtcatccccagtgaaaggtgcttcgtttgtcaccagtaccaatcctcggaaattttctggattttcagccaaaaccaacaactctgagcaaggctccttaagctctgcccctaagacaagtctgtctacaagtaaatgtgaccctaagcacaaagactgtctactacgagagttccggaagctgtgcgccatggtggctgaaaatcctagctacaatacaaagacccagatcatccaggacttcttgcagaaaggctctgcaggagatggcttccacggtgatgtgtacctaacagtgaagctactgctgccgggagtcgttaagagtgtttacaacttgaacgataagcagattgtgaaactttttagccgaatttttaagtgcaacccagatgatatggcccgggacctagaacagggtgacgtatcagagacgatcagagtcttctttgagcagagcaagtctttccccccagctgccaagagcctcctcaccatccaggaagtggatgccttcctcctgcacctctccaagctcaccaaagaggatgagcagcagcaggccctgcaggacattgcctccaggtgtacagccaatgaccttaagtgcatcatccggctgatcaagcatgatctgaagatgaactcgggtgcaaagcatgtgttagatgcccttgaccccaatgcttatgaagccttcaaagcctcgcgaaacctgcaggatgtggtggagcgagtccttcacaatgagcaggaggtgaagtaccaaggccagcgacggactctgagcgttcaggcctcactgatgactcctgtgcagcccatgctggctgaggcctgcaagtccatcgagtatgcaatgaagaagtatcccaatggcatgttctctgagatcaagtacgatggtgagcgagtccaggtgcataagaagggggaccacttcagctacttcagccgcagtctcaagcctgtcctgcctcacaaggtggcccactttaaggactacatccccaaagcctttcctgggggtcagagcatgatcttggactccgaagtgctcctgattgacaacaacactggcaaaccactgccctttgggactctgggagtgcacaagaaagctgccttccaggatgctaatgtttgcctgtttgtttttgattgtatctactttaatgatgtcagcttgatggacaggcctctctgtgagagaaggaagcttcttcacgacaacatggttgaaatccagaaccggatcatgttctcagaaatgaagcaagtcacaaaagcctccgacctggctgacatgataaaccgggtgatccgagagggcttagaagggctggtgctaaaggatgtaaagggtacatacgagcctgggaagcgacactggctgaaagttaagaaagattatttgaacgagggggccatggcagatacagctgatctggtggttcttggggccttctatgggcaagggagcaaaggtggtatgatgtccatcttcctcatgggctgctatgaccctgacagccagaagtggtgcactgtcaccaaatgtgctggaggccacgatgatgccacacttgcccgactgcagaaggagctagatatggtgaagatcagcaaggatcccagcaagatacctagctggctgaagatcaacaagatctactatcctgacttcattgtttcagacccaaagaaagctgctgtgtgggagatcacaggggcagaattttccaaatctgaggctcacactgctgatgggatctccatccgatttcctcgatgcactcgaatccgggacgacaaggactggaaatctgccactaacctcccccaactcaaggaactataccagctgtccaaagaaaaggcagacttcgatgtagtggctggagatgaggggaattccagtaccggaggtagcaatggtgagaatgagggcactgctgggtctgctgcaccccacaaggcccccaaggcacctcctagtaagtcctctgccagtgacaagaaggccaaacagaagctgaataaccccaacagcagagatggcaacaaggtgattccaaagccttcccccatgaaaccaggagacaagctggctatgaagtcttctccagtgaaagtaggggtgaagaggaaagctgctgatgaaacccagtgcccgacaaaggtactgttggatgtcttcactggggtgcggctctacttgcctccttccacaccagacttcaaacagctcaaacgctactttgtggcattcgatggagacctggtacaagaatttgacatggcctccgccacacatgtgctgggtaacagggacgacaacactgaggcccagctggtctctccagagtggatttgggcatgtatccggaaacggaggctgatagctccctgctag

SEQ ID NO: 14 - protein, 1013 aa, Cricetulus griseus
MTLAFKILFPRNLCALGRKELCLFSEQNHWPVIRQFSQWSETDLLRGCCLLQRRKPVLSFQGGHLRPRATHLVFFPGSHVGLYTGPYEMAEQRFCVDYAKRGTAGCKKCKEKILKGVCRIGKVVPNPFSESAGDMKEWYHVKCIFEKLERARATTKKIEDLTELEGWEELEDDEKEQISQHIADLSSKAAGTPKKKTAVQAKVTTTGQVSSPVKGASFVTSTNPRKFSGFSAKTNNSEQGSLSSAPKTSLSTSKCDPKHKDCLLREFRKLCAMVAENPSYNTKTQIIQDFLQKGSAGDGFHGDVYLTVKLLLPGVVKSVYNLNDKQIVKLFSRIFKCNPDDMARDLEQGDVSETIRVFFEQSKSFPPAAKSLLTIQEVDAFLLHLSKLTKEDEQQQALQDIASRCTANDLKCIIRLIKHDLKMNSGAKHVLDALDPNAYEAFKASRNLQDVVERVLHNEQEVKYQGQRRTLSVQASLMTPVQPMLAEACKSIEYAMKKYPNGMFSEIKYDGERVQVHKKGDHFSYFSRSLKPVLPHKVAHFKDYIPKAFPGGQSMILDSEVLLIDNNTGKPLPFGTLGVHKKAAFQDANVCLFVFDCIYFNDVSLMDRPLCERRKLLHDNMVEIQNRIMFSEMKQVTKASDLADMINRVIREGLEGLVLKDVKGTYEPGKRHWLKVKKDYLNEGAMADTADLVVLGAFYGQGSKGGMMSIFLMGCYDPDSQKWCTVTKCAGGHDDATLARLQKELDMVKISKDPSKIPSWLKINKIYYPDFIVSDPKKAAVWEITGAEFSKSEAHTADGISIRFPRCTRIRDDKDWKSATNLPQLKELYQLSKEKADFDVVAGDEGNSSTGGSNGENEGTAGSAAPHKAPKAPPSKSSASDKKAKQKLNNPNSRDGNKVIPKPSPMKPGDKLAMKSSPVKVGVKRKAADETQCPTKVLLDVFTGVRLYLPPSTPDFKQLKRYFVAFDGDLVQEFDMASATHVLGNRDDNTEAQLVSPEWIWACIRKRRLIAPC

SEQ ID NO: 15 - DNA, 2796 bp, Cricetulus griseus
  CDS (1)..(2796): CHO Ligase 1
atgagaaaaaaagaaccagagaggaaaggggagaactctgctgccaccatgcagaaaagtatcaggtcatttttccaacccatgaaagagggtaaagcacagaagccggagaaggagacagctaacagcaccaaagagaaggagccacctccaaaggtggcactgaaggagaggaatcgagcagtgcctgagagtgattctccagtgaagaggcctggaaggaaggcagcccaggtcctaagcagcgaaggggaggaggaagatgaagcccccagcacccctaaagtccagaagtctgtgtcagactccaaacaaagctctcctcccagccctgacgcatgtcctgagaacagtcctttccacagtagcccctccatggagatctccccatcaggattcccgaagcgtcgcactgctcggaagcagctcccgaaacggacaattgaggacactgtggaggagcagaatgaggacaaaggcagagcagccaagaaaaggaagaaggaagaagaagcacagactccaatggaaagcctcacagagagtgaagatgtaaaacccaaggaagaaaaggaggagggcaagcatgctgaggcttccaagtcccctgagtcgggaaccttgacaaagacagagaccatcccagtgtgtaaggccggcgtgaaacagaagcctcaggaagaggagcagagcaagcctcctgccagaggcgccaagacactcagcagcttcttcactccccggaagccagcagagaaagccatagtgaaacaagaagagccaggtactccagggaaggaagagaccaagggagccctggatccaacaaattacaatccttccaagagaaactaccaccccattgaagatgcctgctggaaacatggccagaaagtcccttttctcgctgtggcccggacctttgagaagattgaggaggtttctgctcggctcaagatggtggagacactgagcaacttgctgcgctcggtggtggccctgtcacctccagacctgcttcctgttctttacctcagcctcaaccgccttgggccacctcagcagggactagagctgggtgttggtgatggtgtcctccttaaggcagttgcccaggccacaggccgtcagctggagtccatccgggctgaggtagctgagaagggtgacgtgggactggtggccgagaacagccgcagcactcagagactcatgctgccccctcctccgctcaccacctccggggtctttaccaaattctgtgacattgcccggctcactggcagtgcttccatggccaagaagttggatgtcatcaagggcctgtttgttgcctgccgtcactcggaagcccggttcattgccaggtccctaagtggacgcctgcgcctcgggctggctgagcagtccgtcttggctgcccttgccctggctgtgagcctcacaccccctggccaagaatttcccccagctgttgtggatgctgggaagggcaagaccacagaggccagaaagacatggttggaagaacaaggcatgatcttgaagcagaccttctgtgaggtacctgacctggaccgaatcatcccggtgctgctggaacatggcctggaacgcctcccagagcactgcaggctgagcccaggggtccctcttaaaccaatgctggctcatcccactcggggtgtcagcgaggtactgaaacgctttgaggaggtggactttacctgcgagtacaaatatgacgggcagcgggcccagattcatgttctggaaggtggagaggtgaagatcttcagtaggaaccaggaagacaacacaggaaagtacccggacatcatcagccgcatccccaagattaaactcccctcggtcacctcctttatcctggacactgaggctgtggcctgggaccgggaaaagaagcagatccagccattccaagtgctcaccacacgcaagcgcaaggaggttgacgcctcggagatacaggtgcaggtgtgtctgtatgcctttgatctcatctacctcaacggagagtccctgattcgccagcccctgtctcgacgtcggcagctgctccgggagaactttgtggagacagagggtgagtttgtcttcgccacctccctggacaccaaggacatcgagcagatcgctgagttcttggagcagtccgtgaaggactcctgtgagggactgatggtgaagaccctggatgttgatgccacctatgagattgccaagaggtctcacaactggctcaagctaaagaaggactaccttgacggtgtgggcgacactctggaccttgtggtgattggcgcctacctgggccgggggaagcgtgccggccggtatgggggcttcctcttggctgcctatgatgaggagagtgaagagctgcaggccatatgcaagctgggaactggattcagtgatgaagagctggaggagcatcaccagagcctaaaggccctagtgttgccgaccccacgcccctatgtgaggatcgatggggcagttgccccagaccactggctggacccaaaggtcgtatgggaggtgaagtgtgcggatctctccctgtcccctatctaccctgctgcgcggggcctggtggacaaagagaaagggatctcccttcgtttccctcggttcattcgtgtccgtgaagacaagcagccagagcaggccaccaccagtgaccaggtggcctgtttgtaccggaagcagagtcagatacagaaccagcacaactcagacttggactccgactttgaggactgctattaa

SEQ ID NO: 16 - protein, 931 aa, Cricetulus griseus
MRKKEPERKGENSAATMQKSIRSFFQPMKEGKAQKPEKETANSTKEKEPPPKVALKERNRAVPESDSPVKRPGRKAAQVLSSEGEEEDEAPSTPKVQKSVSDSKQSSPPSPDACPENSPFHSSPSMEISPSGFPKRRTARKQLPKRTIEDTVEEQNEDKGRAAKKRKKEEEAQTPMESLTESEDVKPKEEKEEGKHAEASKSPESGTLTKTETIPVCKAGVKQKPQEEEQSKPPARGAKTLSSFFTPRKPAEKAIVKQEEPGTPGKEETKGALDPTNYNPSKRNYHPIEDACWKHGQKVPFLAVARTFEKIEEVSARLKMVETLSNLLRSVVALSPPDLLPVLYLSLNRLGPPQQGLELGVGDGVLLKAVAQATGRQLESIRAEVAEKGDVGLVAENSRSTQRLMLPPPPLTTSGVFTKFCDIARLTGSASMAKKLDVIKGLFVACRHSEARFIARSLSGRLRLGLAEQSVLAALALAVSLTPPGQEFPPAVVDAGKGKTTEARKTWLEEQGMILKQTFCEVPDLDRIIPVLLEHGLERLPEHCRLSPGVPLKPMLAHPTRGVSEVLKRFEEVDFTCEYKYDGQRAQIHVLEGGEVKIFSRNQEDNTGKYPDIISRIPKIKLPSVTSFILDTEAVAWDREKKQIQPFQVLTTRKRKEVDASEIQVQVCLYAFDLIYLNGESLIRQPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLAAYDEESEELQAICKLGTGFSDEELEEHHQSLKALVLPTPRPYVRIDGAVAPDHWLDPKVVWEVKCADLSLSPIYPAARGLVDKEKGISLRFPRFIRVREDKQPEQATTSDQVACLYRKQSQIQNQHNSDLDSDFEDCY

SEQ ID NO: 17 - DNA, 7716 bp, Cricetulus griseus
  misc_feature (1)..(7716): CHO Polymerase theta
atgagtcttccgcgccggagtgggaaacggcggcgctcggcgtccggctccgactcgttctcgggagacggtgatagctgcgccagccctcagctgccgtctaggccggtgctgagtccaccttcgcggctgggacgcggcccgagagccgcgggtgcagggacatgcaagaaaagagttcctgatgaccagatagaccaactgctattggcaaattggggagatcctaaagcagttctggagaaataccatagttttggtgtaaaaaagatgttcgaatggcaggcagagtgtcttttgcttggacaagtcctggaaggaaagaatctagtttattcagctcctacaagtgccgggaagactcttgttgcagagttacttattttgaagcgtgttttggaaatgcggaagaaagctttgtttattctgccctttgtgtctgtggctaaagagaagaaatactacctccagagtctgtttcaggaagtaggaataaaagtagatggctacatgggtagttcctcacctactggacgcttttcctccttggacattgctgtttgcactattgagagagccaatgggctgatcaatcgcctcattgaggaaaataagatggatttgttaggaatggtggttgtagatgaattacacatgctgggagactctcaccgaggatatcttctggaacttttactgaccaagatttgctatgttactaggaaatcaacattgtgccaggcagattcagcccgtgctttgtgtaatgctgtgcagattgttggcatgagtgctactcttcctaatttgcagcttgtagcttcctggttggatgctgaactttaccatactgactttcgccctgtaccacttttggaatcaataaaaataggaaattccatatatgactcttcaatgaagctcgtgagagaacttcagcctgtgcttcaagtgaagggagatgaagatcacattgttagtttatgttatgagaccgtttgtgataaccattcagtattactcttttgtccatcaaaaaaatggtgtgaaaaggtagcagatatcattgcccgagagttttataatctgcaccatcaacctgagagattagtaaaaccatctgaatttccaccagtgaatctagaccaaaagagccttctggaagtgatggatcagctaaaacgttcgccttcaggactagactccgtattgaagaacactgtgccatggggagtagcatttcatcatgcaggtcttacttttgaggagagagatatcatagaaggagccttccgtcaaggtcttattcgggtcttggcagcaacatctactctttcttctggagtgaatcttcctgctcgccgggtgattattcgcactccagttttcggtggccagactttagatatcctcacttataagcagatggttggccgtgctggcaggaaaggagtagacacaatgggtgaaagtatcctagtttgtaagccctctgagaaatcgaaaggcgttgctctgcttcaaggatctctggaacctgtgcatagctgtctgcagagacaagaagtcactgctaacatgatacgagcgattctggagataattgttggtggagtggcaagtacatcacaagatatgcagacttatgcctcctgcacattcttggctgctgctgtaaaagaagggaagcagggaattcagagaaatcaagatgatgttcattttggagcaattgatgcctgtgtgacatggctgttagaaaatgagttcatccaggaggcagagcccagtgatggctcaggaggaaaggtgtatcacccgacacatcttggatcggctactctttcttcctcactctctccaactgataccctagatatttttgctgacctccaaagagcaatgaaaggatttgttttagagaatgaccttcatatcgtctacctggttacacctgtgtttgaagattggactggtattgattggtatcgatttttctgtttatgggaaaaattgcccacttctatgaaaagggtggcagaactggttggagttgaggaagggttcttggctcgatgtgtgaaaggaaaagtagtagctagaactgacagacagcatcgacaaatggccatccataaaaggtttttcacaagtcttgtgttattggatttaatcagtgaaattcccttaaaggaaattaatcagaaatatggatgtaatcgtggacagattcaatctttgcaacagtcagctgctgtttatgcaggaatgattacagtgttttccaaccgtcttggctggcacaatatggaactactactttcccagtttcagaagcgtcttacctttggcatccagagggaactgtgtgacctcatccgggtgtccttactgaacgcccagagagccaggttcctgtatgcctctggctttctcactgtagcagaccttgccagagctaatgttgcggaggtggaggtggttctgaagaatgctgtgcctttcaaaagtactcgaaaggcagtagatgaagaagaggaagcagccgaagaacgtcgcaatatgcaaaccatttgggtgacgggcagaaaaggtttatctgcaagggaagcagcaaccttaatagtggaagaagccaaaacgattctgcagcaggacttacttgaaatgggagtgcagtgggatccaaattcatccttgagttctagcacatcttcactgaccagtagtgagtcagaagtaaatgaacgcacacttcaatctcaaactaagaattctcataaaaagttgacatcaaagaatagaaacagtgtgagagcaagtgtttctaatgataaaccgtcaccagatacagcacaaggcttaggtgaacacagcgagcatacagactccctctgtttgttacaggggaacaaacaccagcaccagccacattctgtttgcagagcaagaaaacggacttctttgggtataaacaaagaaaagcttcggatgtctctgaatggaggagaaccaagcactaaggaagttcttcaaactttctcaatggagaaaacaaggaaagctgctttgactgctaattcagagcagacaaacaccagtttcccatcttggagagacagaaagcatcgaaaaaaatcttggggcagtagcccagtgcgtgattctagggctaatcatcacagagatgatttccaggaacacactgtgtctagatctactctatgtgaagagccactttccttagacaagcagaatatagaatttagaagttcagggctacttataaaaaatgcatctttttgtgccaatgaaaaatacaacaaaacctcattctctttacaaatgcaacagccttgcctaaggaaaaaaacagaaagtactggtgctgtggaacattcctttgcagaatctcagagtaaaaatgtgactggtcagtcccctggcgttgctagcaatggaagaggattggctgatactgagacaggaaagataaatgaggtactcatagagaatggtgcagaaagtcagaatgtttctgtgaaacaccatgacacccatccaattagccagtgcctggaaaatcagtgtgacaaacagacaaacacttgcactaagcggaaagcattaatagagagacaagtgtcctgtgaagcagttagttacatggccagagactcaaatgatgtttcaactatcaactctgaaagcataaagcttcatagcaaagacgatgagtcaaatcactgtcaggtattgggaaataatacgggcagaagtgaggcacctcgtggattactgcagtcagctgcagaattcagtcaagcagatggccagcatgagcatcttctaaattcttctggaatacaagaaaaaacagatgcttatgccacaaataaaactgaacataatcatgtttctaacttagccccctgtgactttggagatagcttctatctggatactcagtcagagaaaataattgaacagttggcaactgaacatgccaagcaaagaacaaaggcggtaacagcgaagggctctgacactcggaactcagggagctcatttcagaacaagtgtcacagtactcgaggtgaacagcacttccaaagagcagccaacacagaccatttggacagtaagagtgtagagaccacaaaacagaaccctgaaaagagcatcggtagactaactgcagaaagcatcatcttccactctccaacaccacagggggaaaatggcccttgctttagagtgaatgagcagtctgttactgactcccagctaaacagttttcttcaaggttttgaaacacaagaaatggttaaaccagtcctatctctagctcctccagcgggaactcctactggtttggaggaagaaaatctgcctgaaaccagtttgaatatgagtgacagtatactatttgacagttttggtgaagatgacttagtaaaaggacagtcacctgatgtacaggcaaagcagcccctcctttctgtaatgacaccaaaccatctcagcagctctctgtgcccacgagaagacccagtcatgaaggcaaatgtgaatgaccatcagggtattcagcagccggagacttgttccagtggggaatctgttatattttcagaggtggattctgctcagatgattgaagccttggacagtgtggctgcattacatgtccaacagaattgtaactcagtaacccttaagactttagaactcagtgattctgcaatgctggataatgagtgtccccaaggaaaagtggccagaggagataaaagcgaaagggcccaaatgtccaaactcactgagacaaatcaagataattcaatcacttggtcaggagcatcatttgacttaagtccagaactgcaaaggattttagacaaagggtctagtcctctagaaaacgaaaggcctaaattaacacagacaaacttgtcttgctttgaaagaaacggtacagagttaaatgagagacaggaaatgaatccaaatttggaggccgttcaaattcagagaacttcacttttccccaataatggagttcaaaacaagattgaggggatagggaacgacaccaggcgtggtgaggccttatatccctcagctcgtaaagaaagtgacacagctgatgacaatggcctcattcctcccacacccattcttgcttctacttctaagctgtcatttccagaaattctcggaacatctgtaaaccatctgaaagctgacagtgtttttctgccaggtgaaagttgtttatttggctcaccttcagatagtcaagagcgtagagacagcttcaaagatgaccgttcagttggagacacaagtttttctctgcagttctctcaggatgaactgcagctaactccagcctcatgcagctccgaaagcttggccataattgatgtagcgagtgaccacacgctgtttgagacgtttgtgaaggagtggcgatgccaaaagcgcttttccatctctctggcttgtgaaaagattagaagtccgacatcttccaaaaccgctaccatagggggaaggttgaagcaagtgagctcccctcagaagacctctgctgaagatgatggctttcctgtccatggctctgatggtgtcatagtagttggactagcagtgtgctggggtggaagggatgcctattatttgtcactgcagaaggaacaaaagcattccgaaattagccccagtttggccccacctcctctggatacaacattgactgtggaagagaggatggagcaccttcaatcctgcttgcaaaagaaatctgacaaagagcagactgttgtcacctatgacttcatccagagctataaaatcctcctcctgtcctgtggtgtctccctggaaccaagttatgaagatcctaaggtggcatgctggcttctcgatccagactctaaggaaccaactctccatagcatagtgaccagtttcctccccgaggagcttcctctgctagaaggaatagagacaggccaagggattcagagcctggggctaaatgtggacactgagcattctgggcggtacagagcatctgtggaatccattctcatcttcaactccatgaatcaactcaactctttgttgcagaaggaaaaccttcacgatattttctgtaaagtggagatgccctcccagtactgcctggccttgctagaactgaatggaattggctttagtactgcagaatgtgaaagtcagaagcatatcatgcaagccaagctggacgccattgagactcaggcctatcagctagctggtcacagcttttcctttaccagtgcagatgacatcgcacaggttttatttttggagctgaagctaccaccgaatggagagatgaaaatgcaaggcagcaaaaaaactttgggttctaccagaagaggcaatgagaatggccacaaactaaggctgggaagacagttcagcactagcaaggatgttttaaataaattaaaggccttacatcctttaccaggcctgatactggaatggagaagaatcagtaatgctattaccaaagtggtcttccctctgcagcgggaaaagcgcctgaacccttttctcagaatggaaaggctctaccctgtatcacagtcacacacggccacaggacggataacctttatagaacctaatatccagaatgtgccaagagattttgagattaaaatgcccacagtagtaagggaaagcccaccttctcaagctccaggcaaacgcctgcttccaatgaccagaggacaaaataagaagttttatggcttgcaccctgggaatgggacactgatggaagagaaagcctcagatagaggggtgccattttcagtgagcatgcgccatgcttttgtgcctttcccagggggtgtaattttggctgctgattattctcagcttgaactgaggatcctggctcacctgtcacgtgactgtcgcctcattcaagtcttaaacactggagctgatgttttccggagcattgctgctgaatggaagatgatcgagcctgactctgttggggaggatctgaggcaacaagcaaagcagatttgctatggaatcatatatgggatgggagctaaatccttaggagagcagatgggcattaaagaaaacgatgctgcttgctatattgactctttcaagtccagatatacagggattaatcatttcctgagggacacagtgaaaaaatgtagaagagatggatttgttcagaccattttgggaaggcgcagatatttgcctggaatcaaagacaacaatccttatcataaagctcatgctgaacgtcaggccatcaacacaacagtccaaggatcagctgctgatatagtaaaaacagccacagttaacattcagaagcaattagagaccttccattcagccttcaaatcccatggtcatcgggaaagcatgctccagcatggccaaacaggattgttgccaaagaaaaaactgaaagggatgttttgtccaatgagaggaggcttctttatccttcaacttcatgatgagcttttatatgaagtggcagaagaggatgttgttcaggtagctcagattgtcaagaaggaaatggaatgtgctgtaaaactttctgtgaagctaaaagtgaaagtgagaatgggtgccagctggggacagctggaggactttgatgtgtga

SEQ ID NO: 18 - DNA, 1389 bp, Cricetulus griseus
  misc_feature (1)..(1389): CHO polymerase delta 3
  CDS (1)..(1389)
atggcggaacagctgtatctggaaaacatagacgagttcgtcacggaccagaacaagatcgtgacttacaagtggttaagctatacactaggagttcatgttaaccaggcaaaacagatgctctatgaatatgttgaaaggaaacgaaaggaaaattcgggagctcagctacatgttacctacttagtgtctggcagtcttattcagaacggacattcgtgccacaaggttgcagtagtgagagaagataaattggaagcagtgaagtccaagctagctgtgactgccagcgtccatgtgtacagcatccagaaagctatgctaaaggacagtgggcctctgttcaataccgactatgacatccttaaaagcaatttgcagaactgcagcaagtttagtgccatacagtgtgctgctgcggtccccagagctcctgcagagtcctcagcttctagaaagtttgagcaatcaaatcttcaagcagcaagtgtgacacaagccagtgaactgaccaccaatggccatggtccacctgcctccaaacaggcttcccagcagcccaaaggaattatgggaatgttgctctccaaagctgctactaaaacccaagacaccaacaaggaaaccaaagcagaggctaaagaagtaacaaatgcatcttcagctgggggcaaagcaccaggaaaggggaatgtgatgagcaacttttttggaaaagctgctatgaataaacttaaagtcagtttggattcagagcaaggagtgaaagaagaaaaaacagtagagcagcctccagtgtctgtcattgaaccaaagctggcagctcccacagctctgaagaaatccagcaggaaggcagagcctgtgaagatgcagcagaaggaaaaaaaatgggggaagcgagtagacttatctgatgaggaagcaaaggaaaccgaaaacctgaagaaaaagaggagaagaatcaagctccctcagtccgatagcagtgaagatgaagtcttcccagactctcctgagatgtatgaagcagagtcaccatctccacctcctcctgcatctccacctcctgattctatgcctaaaactgagcccccgcctgtcaagagttcaagtggagaaaacaaaagaaaacgaaagcgtgtactgaaatctaaaacctttgtggatgaagagggctgcatagtgactgaaaaagtctacgagagtgaatcgtgcacagacagtgaagaggagagtaagatgaaggtgacatcagtacacagaccccctgctgctactgtgagaaaggagcccaaggaagaacgaaagggccccaagaaagggaccactgctctgggcaaagccaacagacaagtgtccattactggcttcttccagaagaagtaa

SEQ ID NO: 19 - protein, 462 aa, Cricetulus griseus
MAEQLYLENIDEFVTDQNKIVTYKWLSYTLGVHVNQAKQMLYEYVERKRKENSGAQLHVTYLVSGSLIQNGHSCHKVAVVREDKLEAVKSKLAVTASVHVYSIQKAMLKDSGPLFNTDYDILKSNLQNCSKFSAIQCAAAVPRAPAESSASRKFEQSNLQAASVTQASELTTNGHGPPASKQASQQPKGIMGMLLSKAATKTQDTNKETKAEAKEVTNASSAGGKAPGKGNVMSNFFGKAAMNKLKVSLDSEQGVKEEKTVEQPPVSVIEPKLAAPTALKKSSRKAEPVKMQQKEKKWGKRVDLSDEEAKETENLKKKRRRIKLPQSDSSEDEVFPDSPEMYEAESPSPPPPASPPPDSMPKTEPPPVKSSSGENKRKRKRVLKSKTFVDEEGCIVTEKVYESESCTDSEEESKMKVTSVHRPPAATVRKEPKEERKGPKKGTTALGKANRQVSITGFFQKK

SEQ ID NO: 20 - DNA, 1707 bp, Homo sapiens
  misc_feature (1)..(1707): EEPD1 (endonuclease/exonuclease/phosphatase family domain 1)
atggggagcaccctgggctgccaccgctccatccccagggacccctcggacctgtcccatagccgcaagttcagcgcagcctgtaacttcagcaacattctagtgaatcaggagcggctcaacatcaacactgccacggaggaggagctgatgaccctgcctggggtgacgcgtgccgtggcacgcagcatcgtggagtaccgagagtatatcggtggcttcaagaaggtggaggacctggcattggtcagtggtgtaggcgccaccaagctggagcaggtcaagtttgagatctgtgtgagcagcaagggcagctcagcgcagcactctcccagttccctgcggcgggacctgctagcggagcagcagcctcaccacctggccacagctgtgcccctcaccccacgtgttaacatcaacacagccaccccggcccagctcatgagcgtgcgaggcctctcggagaaaatggccctcagcatcgtggacttccgccgtgagcatgggccctttcgcagcgttgaggacctagtgaggatggatggtatcaatgccgccttcctggacaggatccggcaccaggtgtttgctgagaggtccaggcccccatccacccacacgaacgggggactgaccttcaccgccaagcctcacccgagccccacttccctgagcctgcagagtgaggacctggacctgccgccaggggggcccacccagattatctccactcggccgtccgtggaggcctttggaggcacaagggatgggaggcctgtgctgaggctggccacctggaacttgcagggctgttccgtggagaaggccaacaaccccggggtgcgagaggtggtgtgcatgacactcctggaaaacagcatcaagcttctagctgtgcaagaactgcttgacagagaggccttggaaaagttctgcacggagctaaaccagccgaccctgcccaacatccgcaagtggaaggggccccggggatgctggaaggctgttgttgctgagaagccctcgaatcagctccagaagggagctgggtatgcaggattcctatgggacgcggctgccggcatggagctgagagacgcgggttcacaggagagctcgccaagcaacgggcacgggaagctggcgggccccagcccatacctcgggaggttcaaggtgggaagtcacgacctgacccttgttaaccttcacctggcagccctgaccctcctggggagcgagaatcccagcaagaatcacagtgatggccaccggttggcgagctttgcacagaccctacaggaaaccctgaaaggagaaaaggatgtcattatcttaggggattttggccaagggccagacagcaatgactatgatatcctgaggaaagaaaagttccaccacctgatccccgcgcacaccttcaccaacatcagcaccaagaaccctcaaggctcgaagtctctggacaacatctggatcagtaaaagcttaaagaaggttttcacaggtcactgggctgtggtgagagaaggcctcacgaacccttggattccggataactggtcttggggcggggtggcttctgaacactgcccagtgctagccgagttctacactgaaaaggactggagcaagaaggacgcccctcggaacggcagcggggtggccttggagcgaagtgaagccaacatcaagcacgagcga

SEQ ID NO: 21 - protein, 569 aa, Homo sapiens
  MISC_FEATURE (1)..(569): EEPD1 (endonuclease/exonuclease/phosphatase family domain 1)
MGSTLGCHRSIPRDPSDLSHSRKFSAACNFSNILVNQERLNINTATEEELMTLPGVTRAVARSIVEYREYIGGFKKVEDLALVSGVGATKLEQVKFEICVSSKGSSAQHSPSSLRRDLLAEQQPHHLATAVPLTPRVNINTATPAQLMSVRGLSEKMALSIVDFRREHGPFRSVEDLVRMDGINAAFLDRIRHQVFAERSRPPSTHTNGGLTFTAKPHPSPTSLSLQSEDLDLPPGGPTQIISTRPSVEAFGGTRDGRPVLRLATWNLQGCSVEKANNPGVREVVCMTLLENSIKLLAVQELLDREALEKFCTELNQPTLPNIRKWKGPRGCWKAVVAEKPSNQLQKGAGYAGFLWDAAAGMELRDAGSQESSPSNGHGKLAGPSPYLGRFKVGSHDLTLVNLHLAALTLLGSENPSKNHSDGHRLASFAQTLQETLKGEKDVIILGDFGQGPDSNDYDILRKEKFHHLIPAHTFTNISTKNPQGSKSLDNIWISKSLKKVFTGHWAVVREGLTNPWIPDNWSWGGVASEHCPVLAEFYTEKDWSKKDAPRNGSGVALERSEANIKHER

SEQ ID NO: 22 - DNA, 783 bp, Escherichia coli
  misc_feature (1)..(783): Restriction Enzyme PvuI
atgaaaaaaaatagatacgaatcaataattgaaggtattttcttagataaatacgttgatggtaatgatattgttgaatttaatcgtactgatatcatttctaaatcagctgaattagatattaatctgccaaaaaatattggtgatgtaatttattcatttaaatatagagcttcgttacccgtatctataacccaaaaagcacaaaatgggaaggaatgggtaataaaaaatatcgggcgttctttatattgctttcaacaagttaactattcaagaatattacctgatatgatgttatcgacaataaaaataccagattcaacaccaacaatagttgctgagcatgcctttaatgatgaacaagcattattaacaagagtcagatacaatcgattaatcgatatatttacaggtgcagtttgttactcattacaaaatcatctaagaacaacagtgccttctgtaggacaaattgaaactgatgaaatatatgttggtgttgaccgtttgggaagacaatttatttttcctgtgcaagctaaaggaggaaaagatgaattgggtattgttcaaatagaacaagactttctactatgtaggcataaataccccaacttgatttgtagacccatagcaacacaatttatttcgaatgataaaatagccatttttgaatttgtattagaaaataatgaagtaaaaaaattacaagaaaagcattatttattagttggtaaagggcaaattagcgtcgatgagctatccaactacaatttttaa

SEQ ID NO: 23 - protein, 260 aa, Escherichia coli
  MISC_FEATURE (1)..(260): Restriction Enzyme PvuI
MKKNRYESIIEGIFLDKYVDGNDIVEFNRTDIISKSAELDINLPKNIGDVIYSFKYRASLPVSITQKAQNGKEWVIKNIGRSLYCFQQVNYSRILPDMMLSTIKIPDSTPTIVAEHAFNDEQALLTRVRYNRLIDIFTGAVCYSLQNHLRTTVPSVGQIETDEIYVGVDRLGRQFIFPVQAKGGKDELGIVQIEQDFLLCRHKYPNLICRPIATQFISNDKIAIFEFVLENNEVKKLQEKHYLLVGKGQISVDELSNYNF

SEQ ID NO: 24 - DNA, 499 bp, Artificial Sequence
  Synthetic construct
  misc_feature (1)..(423): sequence encoding gRNA for Samhd1 locus (228-269)
  misc_feature (424)..(499): sequence encoding chimeric gRNA scaffold
cccaggtctggaggtcgatggttttagagctagaaatagcaagttaaaataaggctagtccgttatcaacttgaaaaagtggcaccgagtcggtgcttttttgctccgcggcacgagaactcaaagccccggggcctgggtcccacgcggggtcccttacccagggtgccccgggcgctcatttgcatgtcccacccaacaggtaaacctgacaggtcatcgcggccaggtacgacctggcggtcagagcaccaaacatacgagccttgtgatgagttccgttgcatgaaattctcccaaaggctccaagatggacaggaaagggcgcggttcggtcaccgtaagtagaataggtgaaagactcccgtgccttataaggcctgtgggtgacttcttctcaccgggacgtgtgctcctacctaagttttagagctagaaatagcaagttaaaataaggctagtccgttatcaacttgaaaaagtggcaccgagtcggtgc

SEQ ID NO: 25 - DNA, 499 bp, Artificial Sequence
  Synthetic construct
  misc_feature (1)..(423): sequence encoding gRNA directed at Znf292 locus (2231-2272)
  misc_feature (424)..(499): sequence encoding chimeric gRNA scaffold
ggagcttttggagggaccgagttttagagctagaaatagcaagttaaaataaggctagtccgttatcaacttgaaaaagtggcaccgagtcggtgcttttttgctccgcggcacgagaactcaaagccccggggcctgggtcccacgcggggtcccttacccagggtgccccgggcgctcatttgcatgtcccacccaacaggtaaacctgacaggtcatcgcggccaggtacgacctggcggtcagagcaccaaacatacgagccttgtgatgagttccgttgcatgaaattctcccaaaggctccaagatggacaggaaagggcgcggttcggtcaccgtaagtagaataggtgaaagactcccgtgccttataaggcctgtgggtgacttcttctcaccgaaattcagctgccaggtcgagttttagagctagaaatagcaagttaaaataaggctagtccgttatcaacttgaaaaagtggcaccgagtcggtgc

SEQ ID NO: 26 - DNA, 96 bp, Artificial Sequence
  Synthetic construct
  misc_feature (1)..(20): sequence encoding guide RNA for cgLrch2 (5′ target sequence) locus
  misc_feature (21)..(96): sequence encoding the chimeric gDNA scaffold
tactaacttgtggttttctggttttagagctagaaatagcaagttaaaataaggctagtccgttatcaacttgaaaaagtggcaccgagtcggtgc

SEQ ID NO: 27 - DNA, 96 bp, Artificial Sequence
  Synthetic construct
  misc_feature (1)..(20): sequence encoding guide RNA for cgLrch2 (3′ target sequence) locus
  misc_feature (21)..(96): sequence encoding the chimeric gRNA scaffold
aattacatgtcaatgaccgtgttttagagctagaaatagcaagttaaaataaggctagtccgttatcaacttgaaaaagtggcaccgagtcggtgc

SEQ ID NO: 28 - DNA, 20 bp, Cricetulus griseus
  misc_feature (17)..(18): double-stranded break (DBS) site  by CRISPR/CAS9_Cas81
tactaacttgtggttttctg

SEQ ID NO: 29 - DNA, 20 bp, Cricetulus griseus
  misc_feature (17)..(18): double-stranded break (DBS) site  by CRISPR/CAS9_Cas82
aattacatgtcaatgaccgt

(19) United States
(12) Patent Application Publication
(10) Pub. No.: US 2020/0109421 A1
(43) Pub. Date: Apr. 9, 2020

(54) METHODS FOR INTEGRATION OF TRANSGENE DNA

(71) Applicant: Selexis S.A., Plan-les-Ouates (CH)

(72) Inventors: Valerie Le Fourn, Plan-Les-Ouates (CH); Severine Fagete, Plan-les-Ouates (CH); Alexandre Regamey, Plan-les-Ouates (CH); David Calabrese, Plan-les-Ouates (CH); Ghislaine Arib, Plan-les-Ouates (CH); Pierre-Alain Girod, Plan-les-Ouates (CH)

(73) Assignee: Selexis S.A., Plan-les-Ouates (CH)

(21) Appl. No.: 16/585,286

(22) Filed: Sep. 27, 2019

Related U.S. Application Data
(60) Provisional application No. 62/738,392, filed on Sep. 28, 2018.

(51) Int. Cl. C12N 15/90; C12N 5/074; C12N 15/85; C12N 9/22

(52) U.S. Cl. CPC C12N 15/90; C12N 5/0607; A61K 45/06; C12N 9/22; C12N 2800/80; C12N 15/85

(57) ABSTRACT

Disclosed herein are methods of genome alteration, in particular genome editing in eukaryotic cells (e.g., mammalian cells), preferably, but not exclusively the integration of exogenous nucleic acids into the genome of a cell or a population of cells. Such methods include the modulation of cell cycle phases via external conditions such as physical separation, temperature, exposure to certain substances such as cell cycle modulators. Genome alteration is also effected via the use of enzymes such as nucleases and nickases and/or the modulation of DNA repair pathways.

### CROSS REFERENCE TO RELATED APPLICATIONS

This application claims the benefit of U.S. provisional application 62/738,392, filed Sep. 28, 2018, which is incorporated herein by reference in its entirety.

### INCORPORATION OF SEQUENCE LISTING

The sequence listing submitted herewith via the USPTO EFS system named 3024-273-SEQ_LIST_ST25, which is 126 kilobytes (measured in MS-WINDOWS), dated Sep. 27, 2019 is hereby incorporated by reference in its entirety.

### FIELD OF THE INVENTION

The invention is directed at methods of genome alteration, in particular genome editing, in eukaryotic cells (e.g., mammalian cells), preferably, but not exclusively at the integration of exogenous nucleic acids into the genome of a cell or a population of cells. Such methods include the modulation of cell cycle phases via external conditions, the use of nucleic acid altering enzymes and/or modification of DNA repair pathways.

### BACKGROUND

The use of cells to manufacture protein-based therapeutics or biopharmaceuticals is rapidly expanding. Since the first use of Chinese hamster ovary (CHO) cells for recombinant protein expression, production processes for recombinant proteins have steadily improved. Product yield, quality, scalability, reproducibility and stability of protein-producing mammalian cell lines could all be improved in the past (Wurm, 2004). The main factors influencing product yield are the time to accumulate a desired amount of biomass, the process duration, and the specific productivity of the cells. Approaches to improve cell specific productivity have focused on increasing the transgene copy number and preventing silencing of the transgene. However, few studies have focused on the transgenesis process itself.

The publications, including patents and patent publications, referenced in the text and/or the appended bibliography are incorporated herein by reference in their entirety.

Transfection, the introduction of foreign DNA into cells, including mammalian cells, is a widely used technique in the development of modified cell lines such as cells producing recombinant biotherapeutics. However, the majority of transfected cells harbor the plasmid DNA not incorporated into their chromosomes. In those cells, the DNA is able to be transcribed, but cannot be copied and therefore will be degraded over time and diluted during mitosis. Insertion of the plasmid DNA into the genome of host cells is a process which occurs infrequently, resulting in low numbers of stable transfectants. Consequently, generation and isolation of stable clones is a laborious and time-consuming process which is incompatible with high-throughput genome manipulation required for systematic studies.

Furthermore, separating cells carrying the insert DNA, ergo recombinant cells, from the majority of nonrecombinants is laborious and time consuming. If the incidence of integration into the genome is increased this step is simplified.

A change, in particular improvement, of the overall integration efficiency will reduce the number of cell colonies to be screened. The topology of DNA is known to affect transfection efficiency. If supercoiled or open-circular plasmid DNA provides greater transfection efficiency than linear DNA (Cherng et al., 1999), linearization, via restriction enzyme digestion, of circular DNA prior to transfection potentially increases the chance of stable integration (Stuchbury and Munch, 2010). Yet, degradation of linearized DNA by cytosolic endonucleases is responsible for the lower efficiency of transfection by linear DNA. Usually, the foreign DNA is integrated into the genome of the target cell randomly (Murnane, Yezzi, and Young, 1990). Integration into inactive heterochromatin results in little or no transgene expression, whereas integration into active euchromatin frequently allows transgene expression, while random integration often leads to silencing of the transgene. Several strategies have been developed to overcome the negative position effects of random integration: site-specific integration strategies targeting the transgene into transcriptionally active regions of the genome (so called hot-spots) are used but require the expression of integration enzymes or additional sequences on the plasmid and strategies using chromatin remodeling elements in the plasmid which organize the genomic architecture. For instance, epigenetic regulators are used to protect transgenes from negative position effects (Bell and Felsenfeld, 1999) and include boundary or insulator elements, locus control regions (LCRs), stabilizing and antirepressor (STAR) elements, ubiquitously acting chromatin opening (UCOE) elements and matrix attachment regions (MARs). All of these epigenetic regulators have been used for recombinant protein production in mammalian cell lines (Zahn-Zabal et al., 2001; Kim et al., 2004) and for gene therapies (Agarwal et al., 1998; Castilla et al., 1998).

The exact mechanism by which plasmid DNA is integrated is not yet fully understood and remains a matter of research. In viral systems, the foreign DNA is integrated into the host genome via viral integration mechanisms. Generally, plasmid DNA delivered by non-viral methods, on the other hand, is integrated by the cell's machinery itself, via DNA repair and recombination enzymes (Haber, 1999; Mjelle, 2015). Double-strand breaks (DSBs) in chromosomal DNA occur spontaneously during DNA replication as a result of fork collapse/stalling or as a result of head-on collision between the replication fork and the RNA polymerase (Mayan-Santos, 2008; Poli, 2016).

To maintain genome integrity, DSBs must be repaired, for instance to allow the replication fork to restart. Therefore, DSB repair is essential for any cell, since these cytotoxic DNA lesions may cause genome rearrangements, such as deletions, duplications, and translocations. Following such a chromosomal event, the DNA repair machinery of the cell is recruited to promote DNA transactions at the locus, based on several pathways. The DNA recombination pathways, also known as DNA repair pathways (DRPs), are cellular pathways that lead to DNA damage repair, such as the joining of DNA molecule extremities after DSBs, and to the exchange or fusion of DNA sequences between chromosomal and non-chromosomal DNA molecules, such as e.g. the crossing-over of chromosomes at meiosis or the rearrangement of immunoglobulin genes in lymphocytic cells.

In the yeast Saccharomyces cerevisiae, DNA repair enzymes encoded by genes belonging to the RAD51/52 epistasis group repair double-strand breaks by homologous recombination (HR). This process requires homologous DNA sequences, usually present on sister chromatids and on homologous chromosomes in diploids. In mammalian cells, however, non-homologous end joining (NHEJ) is a predominant pathway to repair DSBs (Mjelle, 2015). NHEJ is thought to have a major role throughout the entire cell cycle, while HR is particularly effective in the S phase when the break can be repaired using genetic information from a sister chromatid (Mao, 2008). Importantly, there is an interplay between both pathways as cells made deficient for NHEJ by siRNA-mediated suppression of DNA-PK have stimulated HR (Certo, 2011).

The present teachings described herein will be more fully understood from the following description of various illustrative embodiments, when read together with the accompanying drawings. It should be understood that the drawings and examples below are for illustration purposes only and are not intended to limit the scope of the present teachings. The person skilled in the art is readily able to extrapolate from the specific examples.

### BRIEF DESCRIPTION OF THE FIGURES

FIGS. 1A-1B: Cell cycle histograms of CHO cells.

FIG. 1A shows the CHO cell cycle, notably based on G1 phase, S phase and G2 phase.

FIG. 1B shows how CHO cells can be synchronized with chemical compounds that are added to the medium of the cell culture, by arresting the cell cycle at G1 phase (DMSO), S phase (APH), G1/S phase (MTX), G2/M phase (NOCO).

FIGS. 1C-1D: Flow cytometry distribution of CHO cells after releasing cells from synchronization drugs treatment.

FIG. 1C shows the cell cycle progression after releasing cells from synchronization drugs treatment of CHO cells based on a representative flow cytometry analysis.

FIG. 1D shows the scheme of the cell cycle phase duration of CHO cells.

FIGS. 1E-1F-1G: Effect of CHO cell synchronization on transfectability and Ig-G transgene stable integration.

FIG. 1E shows the evaluation of the percentage of electroporated cells with an eGFP-expressing-vector and the level of fluorescence median intensity (FMI) on cytometer imager.

FIG. 1F and FIG. 1G show the evaluation of the IgG production performance by stable pools and the evaluation of the percentage and the secretion mean intensity of producing cells by Cell Secretion Assay (CSA).

FIGS. 2A-2B: Effect of enzyme addition to CHO transfection on productivity at the transfected cell pool level.

FIG. 2A shows the antibody product titer of CHO cells that was evaluated by ELISA at day 9 of the fedbatch process.

FIG. 2B shows the productivity per cell per day (PCD) of CHO cells that was calculated as function of titer and viable cell density during the fedbatch process.

FIG. 2C: Effect of enzyme addition to CHO transfection on productivity at the clone level

FIG. 2C shows the antibody product titer of 6 clones per enzyme condition that was evaluated by ELISA at day 9 of the fedbatch process.

FIGS. 3A-3B: Effect of the nonhomologous end-joining repair pathway DNA-PK inhibitor Nu7441 on productivity in CHO cells.

FIG. 3A shows the antibody product titer of CHO cells that were treated with NHEJ inhibitor Nu7441 before transfection of the antibody-encoding DNA fragment.

FIG. 3B shows the productivity per cell per day (PCD) of CHO cells that were treated with NHEJ inhibitor Nu7441 before transfection of the antibody DNA fragment.

FIGS. 4A-4B-4C: Impact of cell synchronization on recombinant protein expression using CRISPR/Cas-mediated transgene integration.

FIG. 4A shows the distribution of producing cells (PC) that were synchronized and modified with a CRISPR-Cas system, showing the percentage of the high-, medium- and low-producing subpopulations.

FIG. 4B shows the showed the specific productivity (pg·cell-1·day-1) as mean values of 4 cultivation passages of the stable expressing cell pools.

FIG. 4C shows the fold change of production per cell (PCD) achieved in fed-batch culture of pools, obtained for synchronized cells compared to asynchronized cells.

FIGS. 5A-B-C: Effect of DNA repair pathways chemical modulators on transgene integration.

FIG. 5A shows the percentage of producing cells two days after transfection using a cell secretion assay (CSA). The cells were synchronized prior to transfection, and a drug treatment was applied to inhibit DNA repair mechanisms on freshly transfected cells as indicated.

FIG. 5B depicts histograms that show the percentage and secretion mean intensity (SMI) of total producing cells of the four groups in FIG. 5A two days after selection (AS=asynchonized).

FIG. 5C depicts histograms that show the high-, medium- and low-producing subpopulations of stably expressing cells of the four groups in FIG. 5A ten days after selection.

FIGS. 6A-6B: IgG transfection of G1-synchronized CHO cells in presence of NU7441 and Sbf1 restriction enzyme.

FIG. 6A depicts a histogram that shows cell secretion assay (CSA) as percentage (white bar) and secretion mean intensity (SMI) (grey bar) of total producing cells in cells transfected with a trastuzumab IgG-expressing vector in presence of Sbf1 restriction enzyme and the NHEJ inhibitor—NU7441 (0.4 mM; “NU”).

FIG. 6B depicts a histogram that shows the high-, medium- and low-producing subpopulations of stably trastuzumab-expressing cells of the same cells.

### SUMMARY OF THE INVENTION

Provided are means to alter/facilitate the alternation of the genomic nucleic acid(s) of cell(s). Also provided is a method of introducing at least one alteration into genomic nucleic acid(s) of a cell or a population of cells, the method comprising:
i) conditioning the cell or population of cells to obtain a conditioned cell or population of cells, and/or
ii) introducing into and/or expressing in said cell or population of cells, one or more molecules that introduce DNA double-strand breaks and/or DNA single-strand breaks into said genomic nucleic acid, and/or
iii) modulating one or more DNA Repair Pathways (DRPs) of said cell or population of cells, wherein the genomic nucleic acid(s), upon i), ii) and or iii), may comprise the at least one alteration.

The at least one alteration may be a genomic disruption, such as one or more deletions of one or more endogenous nucleic acid(s) and/or one or more insertions of one or more exogenous nucleic acid(s).

The cell or population of cells may be transfected with the one or more exogenous nucleic acid(s) and the at least one alteration may be an insertion of the one or more exogenous nucleic acids into the genomic nucleic acid(s). The exogenous nucleic acid may be a nucleic acid, such as an DNA encoding a RNA and/or protein of interest. The conditioned cell or population of cells of i) may be subjected to ii) and/or iii) or the cell or population of cells of ii) may be subjected to iii). The conditioning in i) may result in a synchronization of growth of cells in said population of cells, and may preferably be adapted to increase a number of the at least one alteration. The conditioning in i) may comprises:

ia) modulation of the cell cycle of the cell or cells of the cell population, preferably a chemical modulation via a small molecule such as a cell cycle modulator including dimethyl sulfoxide, methotrexate, nocodazole, aphidicolin, hydroxyurea, aminopterin, cytosine arabinoside, thymidine, butyrate, butyrate salt, lovastatin, compactin, mevinolin, mimosine, colchicine, colcemid, razoxane, roscovitine, vincristine, cathinone, pantopon, aminopterin, fluorodeoxyuridine, noscapine, blebbistatin, reveromycin A, cytochalasin D, MG132, RO-3306, or combinations thereof; and/or
ib) temperature-based modulation of the cell cycle of said cell or population of cells, such as keeping the culturing temperature above and/or below a threshold temperature, such as 37° C. and/or alternating between a culturing temperature of above and/or below the threshold temperature; and/or
ic) nutrition-based modulation of the cell cycle of the cell or cells of the cell population of said cell or population of cells including limiting nutrients in a standard culture medium such as one or more amino acids, and/or
id) an optional physical separation of a sub-population of cells from the cell population, such as by cytofluorometry, fluorescence-activated cell sorting, elutriation, centrifugal separation, mitotic shake-off and combinations thereof.

The temperature-based modulation in ib) may comprise providing a culturing temperature of less than 37° C. and greater than 30° C., or providing a culturing temperature of about 4° C. The alternating in ib) may comprise reducing the culturing temperature below the threshold temperature and then increasing the culturing temperature of said cell or population of cells above the threshold temperature or vice versa.

Subsequent to the conditioning in i), a number of cells in the population of cells may be in a cell cycle phase selected from the group of interphase, G0 phase, G0/G1 phase, early G1 phase, G1 phase, late G1 phase, G1/S phase, S phase, G2/M phase, and/or M phase may exceed the number of cells in said phase prior to the conditioning, preferably cells in the G1 phase, cells in the S phase,-cells in the G2 phase. The introduction of the one or more exogenous nucleic acids may take place at a time when said cell or a majority of cells of said population are at the G1, S or G2 phase of the cell cycle.

The one or more molecules in ii) may be protein(s), nucleic acid molecule(s) encoding said protein(s) or combinations thereof. They might, for example be or encode transposases, one or more integrases, one or more recombinases, or one or more nucleases or nickases including engineered nucleases or engineered nickases. The one or more nucleases or nickases may be selected from the group consisting of a homing endonuclease, a restriction enzyme, a zinc-finger nuclease or a zinc-finger nickase, a meganuclease or a meganickase, a transcription activator-like effector nuclease or a transcription activator-like effector nickase, an RNA-guided nuclease or an RNA-guided nickase, a DNA-guided nuclease or a DNA-guided nickase, a megaTAL nuclease, a BurrH-nuclease, a modified or chimeric version or variant thereof, and combinations thereof, in particular a zinc-finger nuclease or a zinc-finger nickase, a transcription activator-like effector nuclease or a transcription activator-like effector nickase, a RNA-guided nuclease or an RNA-guided nickase, wherein the RNA-guided nuclease or an RNA-guided nickase may optionally be part of a CRISPR (Clustered Regularly Interspaced Short Palindromic Repeats)-based system, a restriction enzyme and combinations thereof. The nuclease may degrade the 5′-terminated strand of the DNA break, or may degrade the 3′-terminated strand of the DNA break in particular, may degrade up to 3 nucleotides at the DNA break, may degrade up to 5 nucleotides at the DNA break, and/or may degrade more than 5 nucleotides at the DNA break. The restriction enzyme may or not be sensitive to DNA methylation.

The one or more DRPs in iii) may be selected from the group consisting of resection, canonical homology directed repair (canonical HDR), homologous recombination (HR), alternative homology directed repair (alt-HDR), double-strand break repair (DSBR), single-strand annealing (SSA), synthesis-dependent strand annealing (SDSA), break-induced replication (BIR), alternative end-joining (alt-EJ), microhomology mediated end-joining (MMEJ), DNA synthesis-dependent microhomology-mediated end-joining (SD-MMEJ), canonical non-homologous end-joining repair (C-NHEJ), alternative non-homologous end joining (A-NHEJ), translesion DNA synthesis repair (TLS), base excision repair (BER), nucleotide excision repair (NER), mismatch repair (MMR), DNA damage responsive (DDR), blunt end joining, single strand break repair (SSBR), interstrand crosslink repair (ICL), Fanconi Anemia (FA) Pathway and combinations thereof. The modulation of the one or more DRPs may result in favoring a second DRP or a second set of DRPs over a first DRP or first set of DRPs. The modulation of the one or more DRPs may comprise the modulation of a component involved in said one or more DRPs, wherein the component may preferably be a protein, a protein complex or a nucleic acid molecule encoding the protein or the protein complex and/or may be one or more of components set forth in Table 3. The modulation of said one or more DRPs may comprise a downmodulation of said one or more DRPs in said cell or population of cells, e.g., by contacting said cell or population of cells, with one or more inhibitor(s), such as a chemical inhibitor, of the DRP or a component thereof, inactivating or downregulating the component of the said DRP, and/or mutating one or more genes of the DRP(s) for inhibiting expression or activity of the component of the DRP. The inactivating or downregulating may comprise contacting or expressing in said cell or population of cells, one or more inhibitory nucleic acids such as a miRNA, a siRNA, a shRNA or any combination thereof. The one or more DRPs that are downmodulated may be selected from the group consisting of canonical non-homologous end-joining repair (C-NHEJ), alternative non-homologous end joining (A-NHEJ), homologous recombination (HR), alternative end-joining (alt-EJ), microhomology mediated end-joining (MMEJ), DNA synthesis-dependent microhomology-mediated end-joining (SD-MMEJ) and combinations thereof. Any downmodulation may result in an upmodulation of one or more further DRPs. The one or more DRPs that are downmodulated may be a non-productive pathway or may compete with the one or more further DRPs. For example, the downmodulated DRP may be NHEJ and the upmodulated DRP may be HR or MMEJ. The modulation of said one or more DRPs may also comprise an upmodulation of said one or more DRPs in said cell or population of cells. The upmodulation may comprise:
iia) expressing, including causing overexpression of, one or more components of said DRP in said cell or population of cells,
iib) introducing into said cell or population of cells, the component of the said DRP heterologously,
iic) contacting said cell or population of cells, with one or more modulator, preferably a stimulator, such as a chemical stimulator of the one or more component of the said DRP,
iid) mutating one or more genes of said DRP, wherein said mutating may enhance expression or activity of the one or more component of the said DRP, and optionally a downmodulation in any of the ways described herein. In certain embodiments only one DRP (and no other DRP) is modulated. In other embodiments two or more DRPs are modulated.

The invention is also directed at a cell or population of cells, including a prokaryotic or eukaryotic cell or population of cells that comprises at least one alteration in its genomic nucleic acids(s) and was preferably made by one of the methods described herein. The eukaryotic cell may be a yeast cell, a fungi cell, an algae cell, a plant cell or an animal cell such as a mammalian cell, such as a Chinese Hamster Ovary (CHO) cell or a human cell. The cell or population of cells may comprise an exogenous DNA encoding one of more protein of interest, integrated into the genome following cleavage by the compound introducing a double-strand break or a single-strand break in said cell. The protein of interest may be expressed at a level that exceeds a level of expression attained without i), ii) and/or iii), preferably at least at a twofold, three-fold or four-fold level.

Provided is also a kit comprising:

(i) one or more cell cycle modulators;
(ii) or one or more nucleases or nickases such as engineered nucleases or engineered nickases; and/or
(iii) one or more DRP modulators; and
instructions for using one or more of (i), (ii) and/or (iii) to introduce at least one alteration into a genomic nucleic acid(s) of a cell or a population of cells.

The one or more cell cycle modulators may be dimethyl sulfoxide, methotrexate, nocodazole, aphidicolin, hydroxyurea, aminopterin, cytosine arabinoside, thymidine, butyrate, butyrate salt, lovastatin, compactin, mevinolin, mimosine, colchicine, colcemid, razoxane, roscovitine, vincristine, cathinone, pantopon, aminopterin, fluorodeoxyuridine, noscapine, blebbistatin, reveromycin A, cytochalasin D, MG132, RO-3306 or combinations thereof;

the one or more nuclease may be a CRISPR-based system, TALE nuclease or a restriction enzyme; the one or more DRP modulators downmodulate and/or upmodulate a DRP, such as chemical stimulator(s) including RS-1, IP6 (Inositol Hexakisphosphate), DNA-PK enhancer and combinations thereof or chemical inhibitor(s) including Mirin and derivatives, inhibitors of PolQ, inhibitors of CtIP, RI-1, BO2 and combinations thereof.

Also provided is a cell or a population of cells, comprising:

i) conditioned cell or population of cells,
ii) DNA double-strand breaks and/or DNA single-strand breaks in the genomic nucleic acid, and/or
iii) a modulation of one or more DNA Repair Pathways (DRPs), and wherein the genomic nucleic acid(s), of the cell or cells of the population of cells, may comprise the at least one alteration, preferably an insertion.

### DESCRIPTION OF VARIOUS AND PREFERRED EMBODIMENTS

The definitions herein are provided to aid in describing particular embodiments and are not intended to limit the claimed invention. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The singular terms “a,” “an,” and “the” include plural referents unless the context clearly indicates otherwise. Similarly, the word “or” is intended to include “and” unless the context clearly indicates otherwise.

An alteration of genomic nucleic acid(s) in particular DNA of a cell or a population of cells is an alteration relative to the wild type cell or cell population and includes, but is not limited to, a genomic disruption, such as one or more deletions and/or one or more insertions of one or more exogenous, in particular heterologous nucleic acid(s). The cell and the individual cells of a population of cells is collectively referred to herein as “host cell.”

Genome editing is a location, or at least gene-specific alteration in genomic nucleic acid(s) via a genome (or just “gene”) editing tool such as CRISPR-Cas (clustered regularly interspaced short palindromic repeats and CRISPR-associated protein) system or, more generally, a CRISPR based system or a DNA nuclease-based system. A CRISPR-based system can perform gene editing and involves a guide RNA (gRNA) and a CRISPR enzyme (e.g., Cas9 or Cpf1) which is matched with its targeted site of activity by the gRNA.

The original type II CRISPR system from Streptococcus pyogenes comprises the Cas9 protein and a guide RNA composed of two RNAs: a mature CRISPR RNA (crRNA) and a partially complementary trans-acting RNA (tracrRNA). Cas9 unwinds foreign DNA and checks for sites complementary to a 20 base pair spacer region of the guide RNA. Cas9 targeting has been simplified and most Cas-based systems have been engineered to require only one or two chimeric guide RNA(s) or single guide RNA(s) (chiRNA, often also just referred to as guide RNA or gRNA or sgRNA), resulting from the fusion of the crRNA and the tracrRNA. The spacer region may be engineered as required.

Guide nucleic acids, including gRNAs and gDNAs according to the present invention might be anywhere from 10 nucleotides in length, including 10-50 nucleotides, 10-40, 10-30, 10-20, 15-25, 16-24, 17-23, 18-22, 19-21 and 20 nucleotides.

Transfection as used herein refers to the introduction of nucleic acids, including naked or purified nucleic acids or vectors carrying a specific nucleic acid into cells, in particular eukaryotic cells, including mammalian cells. Any know transfection method can be employed in the context of the present invention. Some of these methods include enhancing the permeability of a biological membrane to bring the nucleic acids into the cell. Prominent examples are electroporation or microporation. The methods may be used by themselves or can be supported by sonic, electromagnetic, and thermal energy, chemical permeation enhancers, pressure, and the like for selectively enhancing flux rate of nucleic acids into a host cell. Other transfection methods are also within the scope of the present invention, such as carrier-based transfection including lipofection or viruses (also referred to as transduction) and chemical based transfection. However, any method that brings a nucleic acid inside a cell can be used. A transiently-transfected cell will carry/express transfected RNA/DNA for a short amount of time and not pass it on. A stably-transfected cell will continuously express transfected DNA and pass it on: the exogenous nucleic acid has integrated into the genome of a cell.

A cell/cell population (the latter is often also referred to as cells of a cell line indicating the homogenous nature of the cells in a cell population) according to the present invention is an eukaryotic, preferably mammalian cell/cell population, such as a non-human mammalian cell, capable of being maintained under cell culture conditions. A non-limiting example of this type of cells are HEK 293 (Human embryonic kidney), Chinese hamster ovary (CHOs) cells and mouse myeloma cells, including NS0 and Sp2/0 cells. Modified versions of CHO cell include CHO-K1 and CHO pro-3. In one preferred embodiment a SURE CHO-M cell™ line (SELEXIS SA, Switzerland) is used.

Cell culture conditions are growth conditions in a cell culture medium such as complete/standard culture medium. As the person skilled in the art will appreciate, standard media vary with the cells used. CDCHO Medium is a standard medium sold by THERMOFISHER Scientific for CHO cells. Amino acids are ingredients of cell culture media. Amino acids essential to the cell cultured must be included in a culture medium as cells cannot synthesize these by themselves. They are required for the proliferation of cells and their concentration determines the maximum achievable cell density. L-glutamine is an essential amino acid for many cells. L-glutamine concentrations for mammalian cell culture media can vary from 0.68 mM in Medium 199 to 4 mM in Dulbecco's Modified Eagles's Medium. Nonessential amino acids may also be added to the medium to replace those that have been depleted during growth. Supplementation of media with non-essential amino acids is known to stimulate growth and prolong the viability of the cells. In certain embodiments, over- or undersupply of an essential or non-essential amino acid can be used/is used to modify the cell growth of a cell or cell population in the medium, including shifting the times in which a cell remains in a certain cell growth phase.

Culturing cells at room temperature signifies that a cell is cultured at temperatures between 18 and 24° C. (degrees Celsius). For mammalian cells the optimal temperature of growth is about 37° C. The present invention includes embodiments in which the temperature of the cell culture medium is less than 37° C. and greater than 30° C., but also between 25° C. and 30° C., between 20° C. and 25° C., between 15° C. and 20° C., between 10° C. and 15° C., between 4° C. and 10° C. or below 30° C., below 25° C., below 20° C., below 15° C., below 10° C., below 5° C., about 4° C. for more than 1, 2, 3, 4, 5, 6, 7, 8, 9, 10, 11, 12, 13, 14, 15, 16, 17, 18, 19, 20, 21, 22, 23, 24, 36, 48 or 72 hours. In certain embodiments the temperatures are switched in one culturing cycle. Thus, for example in an overnight culture with a culturing time of about 18 hours, the cells are grown initially at about 4° C. and, after 9 hours the temperature is switched to between 30 and 37° C. or vice versa. Cycling of the temperature is also within the scope of the present invention so that, for example, the cells that are cultured for a specific culturing time, are for 4 hours cultured at between 30 and 37° C., then cultured for two hours at about 4° C., then switched back to between 30 and 37° C. for 4 hours, followed by two hours at about 4° C. A threshold temperature according to the present invention is for example 37° C., 30° C., 25° C., 20° C., 10° C., 5° C., or 4° C. A population of cells may be synchronized as described elsewhere herein e.g., by combining the use of a cell cycle modulator, such as DMSO, with a certain temperature, such as a temperature of about 4° C.

A vector according to the present invention is a nucleic acid molecule capable of transporting other nucleic acids to which it has been linked. A plasmid is, e.g., a type of vector. In certain aspects of the present invention a vector is used to transport exogenous nucleic acids into a cell or cell population.

### Examples of CRISPR/CAS9 Plasmid-Expression Vectors:

### CRISPR/CAS9 Samhd1:

CRISPR/CAS9_Samhd1 (SAM and HD domain 1) targets the cgSamhd1 gene (cg: Cricetulus griseus). This vector (offered by ATUM) is used for the transient expression of a D10A mutant of Cas9 (Cas9n) that nicks single strands and a pair of offset guide RNAs complementary to opposite strands of a cgSamhd1 locus. Nicking of both DNA strands by a pair of Cas9 nickases leads to a site-specific double stand break (DSB) in the cgSamhd1 locus. The vector is a CRISPR/Cas9-D10A vector derived from the pD1431-Apuro ATUM backbone vector. The sequence encoding the gRNA for the Samhd1 locus (228-269) and the adjoining sequence encoding the chimeric gRNA scaffold is shown in SEQ ID NO:24.

### CRISPR/CAS9 Znf292:

CRISPR/CAS9_Znf292 targets the cgZnf292 gene. This vector (ATUM) is used for the transient expression of a D10A mutant of Cas9 (Cas9n) that nicks single strands and a pair of offset guide RNAs complementary to opposite strands of a cgZnf292 locus. Nicking of both DNA strands by a pair of Cas9 nickases leads to a site-specific double stand break (DSB) in the cgZnf292 locus. The vector is a CRISPR/Cas9-D10A vector derived from the pD1431-Apuro ATUM backbone vector. The sequence encoding the gRNA for the locus (2231-2272) and the adjoining sequence encoding the chimeric gRNA scaffold is shown in SEQ ID NO:25.

### CRISPR/CAS9 Cas81:

CRISPR/CAS9 Cas81 targets the cgLrch2 locus. This vector (ATUM) is used for the transient expression of the Cas9 nuclease and a guide RNA to introduce a double-stranded break (DBS) in the 5′ cgLrch2 locus at position TACTAACTTGTGGTTTTCTG (SEQ ID NO: 28, bolded and underlined: site of the DSB). The sequence encoding the guide RNA for the cgLrch2 (5′ target sequence) locus and the adjoining sequence encoding the chimeric gDNA scaffold is shown in SEQ ID NO: 26.

### CRISPR/CAS9 Cas82:

CRISPR/CAS9_Cas82 targets the cgLrch2 locus. This vector (ATUM) is used for the transient expression of the Cas9 nuclease and a guide RNA to introduce a double-stranded break in the 3′ cgLrch2 locus at position AATTACATGTCAATGAC̅C̅GT (SEQ ID NO: 29, bolded and underlined: site of the DSB). The sequence encoding the guide RNA for cgLrch2 (3′ target sequence) locus and and the sequence encoding the chimeric gDNA scaffold is shown in SEQ ID NO: 27.

A genomic nucleic acid includes for example a eukaryotic host cell's chromosomal DNA, but excludes the host cell's own extrachromosomal elements such as a host cell's plasmids.

A genomic disruption as used herein, refers to additions and/or deletions and may, for example, occur via DNA repair mechanisms.

Exogenous nucleic acid as it is used herein means that the referenced nucleic acid is introduced into the host cell. The source of the exogenous nucleic acid may be, for example, a homologous or heterologous nucleic acid that expresses, e.g. a protein of interest. Correspondingly, the term endogenous refers to a nucleic acid molecule that is already present in the host cell. The term heterologous nucleic acid refers to a nucleic acid molecule derived from a source other than the species of the host cell, whereas homologous nucleic acid refers to a nucleic acid molecule derived from the same species as the host cell. Accordingly, an exogenous nucleic acid according to the invention can utilize either or both a heterologous and/or a homologous nucleic acid. For example a cDNA of a human interferon gene is a heterologous exogenous nucleic acid in a CHO cell, but a homologous exogenous nucleic acid in a HeLa cell. The exogenous nucleic acid may be part of a vector when introduced into the cell or may be introduced as naked nucleic acid.

In a preferred embodiment the alteration is the insertion of an exogenous nucleic acid, such as a DNA, in particular a cDNA, encoding a RNA and/or protein of interest. The exogenous nucleic acid is generally more than 3 nucleic acids molecules in length, generally more than 10, 20, 30, 40, 50, 60, 70, 80, 90, 100, 110, 120, 130, 140, 150, 160, 170, 180, 190, 200 and is preferably one or more transgenes. Transgenes are exogenous nucleic acids that encode a protein of interest or a functional part thereof. As used herein protein refers generally to peptides and polypeptides having more than about ten amino acids. Proteins of interest are usually expressed by exogenous nucleic acids. However, an exogenous nucleic acid might also induce the overexpression of an endogenous nucleic acid that is of interest. As for the nucleic acids, the proteins may be homologous or heterologous to the host cell. The protein may be produced as an insoluble aggregate or as a soluble protein in the periplasmic space or cytoplasm of the cell, or in the extracellular medium. Examples of proteins of interest include hormones such as growth hormone or erythropoietin (EPO), growth factors such as epidermal growth factor, analgesic substances like enkephalin, enzymes like chymotrypsin, receptors to hormones or growth factors, antibodies and include as well proteins usually used as a visualizing marker e.g. green fluorescent protein. After the stable insertion of one or more exogenous nucleic acids, such as transgenes into the genome of the host cell, the protein of interest is expressed by the cell or that population of cells at a higher yield. A cell having stably integrated an exogenous nucleic acid into this genome is called a recombinant cell.

A transgene is used herein to refer to a DNA sequence encoding a product of interest, also referred to as “transgene expression product” Often such a transgene encodes a protein of interest.

### Conditioning

Cells are conditioned if they have been exposed to one or more specific conditions. The process of subjecting the host cell to such a specific condition is called conditioning. A cell or populations thereof that have been exposed to such specific condition(s) are referred to herein as conditioned cells and conditioned populations of cells. The conditioning might be for up to 1, 2, 3, 4, 5, 6, 7, 8, 9, 10, 11, 12, 13, 14, 15, 16, 17, 18, 24, 48, 72, 86 hours or more. The one or more specific conditions are in particular aimed at committing cells to integrate exogenous nucleic acids, in particular naked DNA, but also DNA integrated into a vector, such as heterologous or homologous transgenes into the chromosomes by recombination at a frequency that is higher by comparison to cells that have not been subjected to the same condition(s). Conditioning includes, but is not limited to:
physical separation of cells of the cell population, such as cytofluorometry, fluorescence-activated cell sorting, elutriation, centrifugal separation, mitotic shake-off and combinations thereof;
modulation of the cell cycle of the cell or cells of the cell population, preferably a chemical modulation via a small molecule such as a cell cycle modulator including dimethyl sulfoxide (DMSO), methotrexate (MTX), nocodazole, aphidicolin, hydroxyurea, aminopterin, cytosine arabinoside, thymidine, butyrate, butyrate salt, lovastatin, compactin, mevinolin, mimosine, colchicine, colcemid, razoxane, roscovitine, vincristine, cathinone, pantopon, aminopterin, fluorodeoxyuridine, noscapine, blebbistatin, reveromycin A, cytochalasin D, MG132, RO-3306, or combinations thereof;
temperature based modulation of the cell cycle of said cell or population of cells, such as keeping the culturing temperature above and/or below a threshold temperature, such as 37° C. and/or alternating between a culturing temperature of above and/or below the threshold temperature; and/or
nutrition based modulation of the cell cycle of the cell or cells of the cell population of said cell or population of cells including limiting nutrients in a standard culture medium such as one or more amino acids.

Cell cycle modulator, as used herein, refers to any compound that regulates progression, notably the physiological and morphological progression, of the cell cycle, and the associated processes of transcription, differentiation, senescence and apoptosis. For instance, a cell cycle modulator can refer to an agent such as a chemical compound that causes a cell to cease dividing and to remain in a defined characteristic phase of the cell cycle. Some cell cycle modulators that may be used in the present context include, but are limited to dimethyl sulfoxide, methotrexate, nocodazole, aphidicolin, hydroxyurea, aminopterin, cytosine arabinoside, thymidine, butyrate, butyrate salt, lovastatin, compactin, mevinolin, mimosine, colchicine, colcemid, razoxane, roscovitine, vincristine, cathinone, pantopon, aminopterin, fluorodeoxyuridine, noscapine, blebbistatin, reveromycin A, cytochalasin D, MG132 and/or RO-3306. Cell cycle modulators that can put at least one cell into a common cell cycle phase with another cell are also called “synchronizing agents.”

In certain embodiments of the conditioning, cell cycle modulators are used to arrest cell growth including the cell cycle of a cell (sometimes referred to as a chemical blockade, or chemical blocking). For instance, metabolic reactions of the cell such as DNA synthesis can be inhibited and/or the cell is arrested, at least for a prolonged time, in a certain cell cycle phase, such as the G1, S or G2 phase (see further discussion below), generally while the entire cell cycle is extended, e.g., by at least 20%, 25%, 50%, 75%, 100% or 150%.

A chemical stimulator, as used herein, refers to a chemical compound that can be used to enhance the expression of a gene or the activity of a protein. As the person skilled in the art will readily recognize, the chemical stimulator will depend which component of which DPR (DNA Repair Pathway) is stimulated. For example, RS-1, a RAD51 stimulator stimulates HR. IP6 (Inositol Hexakisphosphate, DNA-PK enhancer are NHEJ stimulators (see, e.g., Hanakahi 2000, Ma 2002, Cheung 2008).

A chemical inhibitor, as used herein, refers to a chemical compound that can be used to inhibit the expression of a gene or the activity of a protein. As the person skilled in the art will also readily recognize, the chemical inhibitor will depend which component of which DPR is stimulated. Examples of chemical inhibitors of MMEJ include, but are not limited to MRE11 inhibitors such as Mirin and derivatives (Shibata et al, Molec. Cell (2014) 53:7-18), inhibitors of PolQ, inhibitors of CtIP (Sfeir and Symington, “Microhomology-Mediated End Joining: A Back-up Survival Mechanism or Dedicated Pathway?” Trends Biochem Sci (2015) 40:701-714). Examples of HR inhibitors: RI-1 (RAD51 Inhibitor 1) and BO2 (3-(Phenylmethyl)-2-[(1E)-2-(3-pyridinyl)ethenyl]-4(3H)-quinazolinone). See also US Patent Pubs. 2019/0194694A1 and 2015/0361451A1.

In certain embodiments the effect of the conditioning may be further enhanced by introducing into/expressing in the cells or population of cells molecules that introduce DNA double strand breaks and/or DNA single strand breaks such as, but not limited to, nucleases.

The conditioning alone or combined with other processes described herein are designed to and do in a majority of cells in a population change the state of the progression, notably the physiological and morphological progression, of a cell cycle, and/or associated processes of transcription, differentiation, senescence and apoptosis of a cell or population of cells (the state of progression may be referred to herein collectively as the “cell growth state”). Synchronizing is the process of putting cells that were previously not in the same cell growth state into the same cell growth state. For example, as a result of the conditioning the cell or cells in the population of cells may be or may be put into or arrested in a cell cycle phase selected from the group of: interphase, G0 phase, G0/G1 phase, early G1 phase, G1 phase, late G1 phase, G1/S phase, S phase, G2/M phase, and/or M phase. As a result, the number of cells in a specific phase may exceed the number of cells in said phase prior to the conditioning. Subjecting cells to a treatment designed to putting or putting them into a common cell cycle phase is called synchronization. Those cells are said to be “synchronized.” In a preferred embodiment as a result of the synchronization more than 10%, 20%, 30%, 40%, 50%, 60%, 70%, 80%, 90%, 95%, 98% or 100% of the cells in the population are in a particular phase and/or the length for which a cell stays in a particular phase increases, for example at least doubles and/or is now more than 2, 3, 4, 5, 6, 7, 8, 9, 10, 11, 12, 13, 14, 15, 16, 17, 18, 19, 20, 21, 22, 23, 24 hours in the particular preferred phase. Preferred phases are the G1 phase, the S phase and/or the G2 phase. In contrast the time the cell spends in a less desirable phases is reduced to less than 5, 4, 3, 2, 1 hour(s) or less than 30, 20 or 10 minutes.

TABLE 1
Examples of cell cycle modulators
(synchronization agents) employed

| | | | Incubation | |
|---|---|---|---|---|
| Agent | Target | Conc. | time | |
| Dimethyl sulfoxide | — | 1% | 3 | days |
| (DMSO) | | | | |
| Methotrexate (MTX) | dihydrofolate reductase | 1 μM | 18 | h |
| | (DHFR) inhibitor | | | |
| Nocodazole | tubulin depolymerization | 1 μM | 18 | h |
| | (Mitosis inhibitor) | | | |
| Aphidicoline | DNA polymerase α inhibitor | 1 μM | 18 | h |

### Double/Single Strand Breaks

Different molecules are able to introduce double and/or single strand breaks into genomic nucleic acids. The nucleases or nickases of the present invention include, but not limited to, homing endonucleases, restriction enzymes, zinc-finger nucleases or zinc-finger nickases, meganucleases or meganickases, transcription activator-like effector (TALE) nucleases or TALE nickases, guided, in particular nucleic acid guided nucleases or nickases, such as a RNA-guided nucleases or RNA-guided nickases, DNA-guided nucleases, such as the Argonaute (NgAgo) of Natronobacterium gregoryi or DNA-guided nickases, a megaTAL nuclease, a BurrH-nuclease, a modified or chimeric version or variant thereof, and combinations thereof. The RNA-guided nuclease or the RNA-guided nickase are optionally part of a CRISPR-based system.

In a preferred embodiment, these double and/or single strand breaks are introduced by one or more nucleases or nickase. Nucleases can introduce double and/or single strand breaks. The term nickase is reserved to molecules that introduce single strand breaks and may be a nuclease with a partially inactive DNA cleavage domain. For example, nuclease domains of the nucleases may be mutated independently of each other to create DNA “nickases” capable of introducing a single-strand cut with the same specificity as the respective nuclease. With the limitations mentioned herein the following discussions about nucleases equally apply to nickases.

Nucleases are capable of cleaving phosphodiester bonds between monomers of nucleic acids. Many nucleases participate in DNA repair by recognizing damage sites and cleaving them from the surrounding DNA. These enzymes may be part of complexes. Exonucleases are nucleases that digest nucleic acids from the ends. Endonucleases, which are preferred in the present context, are nucleases that act on central regions of the target molecules. Deoxyribonuclease act on DNAs and ribonucleases act on RNA. Many nucleases involved in DNA repair are not sequence-specific. In the present context, however, sequence-specific nucleases are preferred. In one preferred embodiment, sequence-specific nuclease(s) is/are specific for fairly large stings of nucleotides in the target genome, such as 5 and more nucleotides, or 10, 15, 20, 25, 30, 35, 40, 45 or even 50 or more nucleotides, the ranges of 5-50, 10-50, 15-50, 15-40, 15-30 as target sequences in the target genome are preferred in certain embodiments. The larger such a “recognition sequence” the fewer target sites are in a genome and the more specific the cut the nucleases or nickases make into the genome is, ergo the cuts become site specific. A site-specific nuclease has generally less than 10, 5, 4, 3, 2 or just a single (1) target site in a genome. Nucleases that have been engineered for altering genomic nucleic acid(s), including by cutting specific genomic target sequences, are referred to herein as engineered nucleases. CRISPR-based systems are one type of engineered nuclease(s). However, such an engineered nuclease can be based on any nuclease described herein. In one preferred embodiment, the codon(s) of the respective nuclease(s) are optimized for expression in, eukaryotic cells, e.g., mammalian cells. The nucleases/systems of the present invention may also comprise one or more linkers and/or additional functional domains, e.g. an end-processing enzymatic domain of an end-processing enzyme that exhibits 5-3′ exonuclease or 3-5′ exonuclease or other non-nuclease domains, e.g. a helicase domain.

Restriction enzymes are sequence specific nucleases that often are specific for fairly small strings of nucleotides, ergo that have a short recognition sequence. The first letter of the name comes from the genus and the second two letters come from the species of the prokaryotic cell from which they were isolated. For example, EcoRI stems from Escherichia coli RY13 bacteria. Many restriction enzymes are restriction endonucleases and introduce, e.g., a blunt or staggered cut(s), into the middle of a nucleic acid. Many restriction enzymes are sensitive to the methylation states of the DNA they target. Cleavage may be blocked, or impaired, when a particular base in the enzyme's recognition site is modified.

Examples of methylation-sensitive restriction enzymes important in epigenetics include, DpnI and DpnII which are sensitive for N6-methyladenine detection within GATC recognition site and HpaII and MspI which are sensitive for C5-methylcytosine detection within CCGG recognition site.

Some exemplary restriction enzymes used in the examples are listed in Table 2, together with their recognition site, their CpG methylation sensitivity and the number of target sites found in the CHO genome of reference.

TABLE 2
Examples of Restriction Enzymes and
their target sites in the CHO genome

| | Recognition sequence | CpG Methylation | Number of |
|---|---|---|---|
| Enzyme | in CHO genome | sensitivity | target sites |
| Pvul | 5′ . . . CG AT^{▾}CG . . . 3′ | Blocked | 11′605 |
| | 3′ . . . GC_{▴}TA GC . . . 5′ | | |
| Sbfl | 5′ . . . CC TGCA^{▾}GG . . . 3′ | — | 70′162 |
| | 3′ . . . GG_{▴}ACGT CC . . . 5′ | | |
| Ascl | 5′ . . . GG^{▾}CGCG CC . . . 3′ | Blocked | 3′901 |
| | 3′ . . . CC GCGC_{▴}GG . . . 5′ | | |
| BstBl | 5′ . . . TT^{▾}CG AA . . . 3′ | Blocked | 105′498 |
| | 3′ . . . AA GC_{▴}TT . . . 5′ | | |

Endonucleases recognizing sequences larger than 12 base pairs are called meganucleases. Meganucleases/-nickases are endodeoxyribonucleases characterized by a large recognition site (double-stranded DNA sequences of, e.g., 12 to 40 base pairs, such as 20-40 or 30-40 base pairs); as a result this site might only occur once in any given genome.

“Homing endonuclease” are a form of meganucleases and are double stranded DNases that have large, asymmetric recognition sites and coding sequences that are usually embedded in either introns or inteins. Homing endonuclease recognition sites are extremely rare within the genome so that they cut at very few locations, sometimes a singular location within in the genome (WO2004067736, see also U.S. Pat. No. 8,697,395 B2).

Zinc-finger nucleases/-nickases (ZFNs) are artificial restriction enzymes generated by fusing zinc finger DNA-binding domains to a DNA-cleavage domain. Zinc finger domains can be engineered to target specific desired DNA sequences. ZFNs as described, for instance, by Urnov F., et al. (Highly efficient endogenous human gene correction using designed zinc-finger nucleases (2005) Nature 435:646-651) Transcription activator-like effector (TALE) nucleases/-nickases are restriction enzymes that can be engineered to cut specific sequences of DNA. Transcription activator-like effectors (TALEs) can be engineered to bind to practically any desired DNA sequence, so when combined with a DNA-cleavage domain, DNA can be cut at specific locations. TALE-Nuclease as described, for instance, by Mussolino et al. (A novel TALE nuclease scaffold enables high genome editing activity in combination with low toxicity (2011) Nucl. Acids Res. 39(21):9283-9293).

RNA-guided nucleases/-nickases, in particular endonucleases include, for example Cas9 or Cpf1. The CRISPR system has been described in detail. Any CRISPR based system is part of the present invention. In case another RNA-guided endonuclease(s) is/are used, an appropriate guide-RNA, sgRNA or crRNA or other suitable RNA sequences that interacts with the RNA-guided endonuclease and targets to a genomic target site in the genomic nucleic acid can be used.

In certain preferred embodiments, the nuclease is a RNA-guided nuclease. Non-limiting examples of RNA-guided nucleases, including nucleic acid-guided nucleases, for use in the present disclosure include, but are not limited to, CasI, CasIB, Cas2, Cas3, Cas4, Cas5, Cas6, Cas7, Cas8, Cas9 (also known as CsnI and CsxI2), Cas10, CasX, CasY, Cpf1, CsyI, Csy2, Csy3, CseI, Cse2, CscI, Csc2, Csa5, Csn2, Csm2, Csm3, Csm4, Csm5, Csm6, CmrI, Cmr3, Cmr4, Cmr5, Cmr6, CsbI, Csb2, Csb3, CsxI7, CsxI4, CsxIO, CsxI6, CsaX, Csx3, CsxI, CsxI5, CsfI, Csf2, Csf3, Csf4, Cms1, homologues thereof, orthologues thereof, or modified versions thereof, MAD7 such as MADzymes (INSCRIPTA), C2c1, C2c2, C2c3.

In certain preferred embodiments, the nuclease is a DNA-guided nuclease. An “DNA-guided nuclease” refers to a system comprising a DNA guide (gDNA) and an endonuclease. The DNA guide, such as a 5′-phosphorylated single-stranded DNA (ssDNA) guides endonuclease to cleave double-stranded DNA targets within DNA-guided nickase. An “Argonaute-based system” refers to a DNA-guided nuclease based on a single-stranded DNA guide (gDNA) and an endonuclease from the Argonaute (Ago) protein family. The gDNA targets the endonuclease to a specific DNA sequence resulting in sequence-specific DNA cleavage. Ago proteins can be altered via mutagenesis to have improved activity at 37° C. Several Argonaute proteins were characterized from Natronobacterium gregoryi (NgAgo, see, e.g., Gao et al., DNA-guided genome editing using the Natronobacterium gregoryi Argonaute, Nature Biotechnology, published online May 2, 2016), Rhodobacter sphaeroides (RsAgo, see, e.g., Olivnikov et al.), Thermo thermophiles (TtAgo, se e.g. Swarts et al (2014), Nature 507(7491): 258-261), Pyrococcus furiosus Argonaute (PfAgo).

The use of an Argonaute-based system allows for targeted cleavage of genomic DNA within cells.

“TtAgo” is a prokaryotic Argonaute protein thought to be involved in gene silencing. TtAgo is derived from the bacteria Thermus thermophilus. (See, e.g., Swarts et al, ibid, G. Sheng et al, (2013) Proc. Natl. Acad. Sci. U.S.A. III, 652).

One of the most well-known prokaryotic Ago protein is the one from T. thermophilus (TtAgo; Swarts et al. ibid). This “guide DNA” bound by TtAgo serves to direct the protein-DNA complex to bind a Watson-Crick complementary DNA sequence in a third-party molecule of DNA. Once the sequence information in these guide DNAs has allowed identification of the target DNA, the TtAgo-guide DNA complex cleaves the target DNA. Such a mechanism is also supported by the structure of the TtAgo-guide DNA complex while bound to its target DNA (G. Sheng et al, ibid). Ago from Rhodobacter sphaeroides (RsAgo) has similar properties (ibid).

Exogenous guide DNAs of arbitrary DNA sequences can be loaded onto the TtAgo protein (Swarts et al. ibid.). Since the specificity of TtAgo cleavage is directed by the guide DNA, a TtAgo-DNA complex formed with an exogenous, investigator-specified guide DNA will therefore direct TtAgo target DNA cleavage to a complementary investigator-specified target DNA. In this way, one may create a targeted double-strand break in DNA. Use of the TtAgo-guide DNA system (or orthologous Ago-guide DNA systems from other organisms) allows for targeted cleavage of genomic DNA within cells. Such cleavage can be either single- or double-stranded. For cleavage of mammalian genomic DNA, it would be preferable to use of a version of TtAgo codon optimized for expression in mammalian cells. Further, it might be preferable to treat cells with a TtAgo-DNA complex formed in vitro where the TtAgo protein is fused to a cell-penetrating peptide. Ago-RNA-mediated DNA cleavage could be used to effect a panopoly of outcomes including gene knock-out, targeted gene addition, gene correction, targeted gene deletion using techniques standard in the art for exploitation of DNA breaks.

Illustrative examples of Argonaute-based systems and design of gDNAs are disclosed in WO 2017/107898, CN105483118, WO 2017/139264, U.S. Patent Application Nos. 2017367280 and 20180201921, and references cited therein, all of which are incorporated herein by reference in their entireties. An Argonaute-based system optionally comprises one or more linkers and/or additional functional domains, e.g. an end-processing enzymatic domain of an end-processing enzyme that exhibits 5-3′ exonuclease or 3-5′ exonuclease or other non-nuclease domains, e.g. a helicase domain.

A “megaTAL nuclease/-nickase” refers to an engineered nuclease comprising an engineered TALE DNA-binding domain and an engineered meganuclease or an engineered homing endonuclease. TALE DNA-binding domains can be designed for binding DNA at almost any locus of a nucleic acid sequence in a genome, and cleave the target sequence if such a DNA-binding domain is fused to an engineered meganuclease. Illustrative examples of megaTAL nuclease and design of TALE DNA-binding domains are disclosed in described, for instance by Boissel et al. (MegaTALs: a rare-cleaving nuclease architecture for therapeutic genome engineering (2013), Nucleic Acids Research 42 (4):2591-2601), and references cited therein, all of which are incorporated herein by reference in their entireties. A megaTAL nuclease optionally comprises one or more linkers and/or additional functional domains, e.g. a C-terminal domain (CTD) polypeptide, a N-terminal domain (NTD) polypeptide, an end-processing enzymatic domain of an end-processing enzyme that exhibits 5-3′ exonuclease or 3-5′ exonuclease, or other non-nuclease domains, e.g. a helicase domain.

A “TALE DNA binding domain” is the DNA binding portion of transcription activator-like effectors (TALE or TAL-effectors), which mimics plant transcriptional activators to manipulate the plant transcriptome (see e.g., Kay et al., 2007. Science 318:648-651). TALE DNA binding domains contemplated in particular embodiments are engineered de novo or from naturally occurring TALEs, and include, but are not limited to, AvrBs3 from Xanthomonas campestris pv. vesicatoria, Xanthomonas gardneri, Xanthomonas translucens, Xanthomonas axonopodis, Xanthomonas perforans, Xanthomonas alfalfa, Xanthomonas citri, Xanthomonas euvesicatoria, and Xanthomonas oryzae and brgl 1 and hpxl7 from Ralstonia solanacearum. Illustrative examples of TALE proteins for deriving and designing DNA binding domains are disclosed in U.S. Pat. No. 9,017,967, and references cited therein, all of which are incorporated herein by reference in their entireties.

A “BurrH-nuclease” refers to a fusion protein having nuclease activity, that comprises modular base-per-base specific nucleic acid binding domains (MBBBD). These domains are derived from proteins from the bacterial intracellular symbiont Burkholderia Rhizoxinica or from other similar proteins identified from marine organisms. By combining together different modules of these binding domains, modular base-per-base binding domains can be engineered for having binding properties to specific nucleic acid sequences, such as DNA-binding domains. Such engineered MBBBD can thereby be fused to a nuclease catalytic domain to cleave DNA at almost any locus of a nucleic acid sequence in a genome. Illustrative examples of BurrH-nucleases and design of MBBBDs are disclosed in WO 2014/018601 and US2015225465 A1, and references cited therein, all of which are incorporated herein by reference in their entireties. A BurrH-nuclease optionally comprises one or more linkers and/or additional functional domains, e.g. an end-processing enzymatic domain of an end-processing enzyme that exhibits 5-3′ exonuclease or 3-5′ exonuclease or other non-nuclease domains, e.g. a helicase domain.

Other enzymes known to be involved in genome alterations such as transposases or integrases may also be used in the context of the present invention to achieve genome alterations.

“DNA Repair Pathway” or “DRP”, as used herein, refers to the cell mechanisms allowing a cell to maintain its genome integrity and its function, in response to the detection of DNA damages, such as single or double-strand breaks. Depending on several parameters such as the type and the length of DNA damages or the phase in which the cell is at the moment of the said damages, DRPs refer to but are not limited to resection, canonical homology directed repair (canonical HDR), homologous recombination (HR), alternative homology directed repair (alt-HDR), double-strand break repair (DSBR), single-strand annealing (SSA), synthesis-dependent strand annealing (SDSA), Break-induced replication (BIR), alternative end-joining (alt-EJ), microhomology mediated end-joining (MMEJ), DNA synthesis-dependent microhomology-mediated end-joining (SD-MMEJ), non-homologous end joining pathways such as canonical non-homologous end-joining (C-NHEJ) repair, alternative non-homologous end joining (A-NHEJ) pathway, translesion DNA synthesis (TLS) repair, base excision repair (BER), nucleotide excision repair (NER), mismatch repair (MMR), DNA damage responsive (DDR), Blunt End Joining, single strand break repair (SSBR), interstrand crosslink repair (ICL) and Fanconi Anemia pathway (FA). A DRP of the present invention is, however, preferably selected from the group enumerated above.

DNA repair pathways can be inhibited, or rather favored/enhanced. Genes, mRNA or corresponding proteins involved in such pathways can be modulated for inhibiting or favoring/enhancing a pathway (see examples in Table 3).

TABLE 3
DNA Repair Pathways and genes involved

| DNA Repair pathway | Gene |
|---|---|
| resection, NHEJ, HR, MMEJ, SSA | Mre11 |
| resection, NHEJ, HR, MMEJ, SSA | Rad50 |
| resection, NHEJ, HR, MMEJ, SSA | Nbs1 |
| resection, HR, MMEJ, SSA | CtIP |
| resection, HR, NHEJ, FA | BRCA1 (FANCS) |
| resection, HR, NHEJ, MMEJ, SSA, MMR | Exo1 |
| Resection | RECQ1 |
| resection, HR, MMEJ, SSA | BLM |
| Resection | WRNa |
| Resection | RTSa |
| Resection | RECQ5 |
| Resection | Dna2 |
| Resection, NHEJ, HR | 53BP1 |
| Resection | EEPD1 |
| NHEJ | Xrcc4 |
| NHEJ | Ku70 |
| NHEJ | Ku80 |
| NHEJ, MMEJ | LigIV |
| NHEJ | DNA-PKcs |
| NHEJ, MMEJ | XRCC1 |
| NHEJ, MMEJ, BER | PARP1 |
| NHEJ | PARP2 |
| NHEJ | LigIII |
| NHEJ | Artemis |
| NHEJ | PNK |
| NHEJ | TDT |
| NHEJ | Pol μ (mu), POLM |
| NHEJ | Pol λ (lambda), POLL |
| NHEJ | XLF/Cernunnos |
| NHEJ | PAXX |
| NHEJ | TDP |
| NHEJ | APTX |
| NHEJ | WRN |
| NHEJ | RTEL1 |
| NHEJ | CYREN |
| NHEJ | APLF |
| HR | MDC1 |
| HR | Abraxas |
| HR, MMEJ | ATM |
| HR | Bard1 |
| HR, NHEJ | BRCA2 |
| HR | BRCC36 |
| HR | Cyclin D1 |
| HR | CK2alpha |
| HR | CK2beta |
| HR | DNA2 |
| HR | DNAPd |
| HR | DNAPh |
| HR | EME1 |
| HR, MMEJ, SSA, NER | ERCC1 |
| HR, NER, FA | ERCC4 (FANCQ) |
| HR, FA | FANCD1 |
| HR, FA | FANCD2 |
| HR | FANCF |
| HR | FANCM |
| HR | GEN1 |
| HR, NHEJ, MMEJ, SSA | MRE11 |
| HR | MUS81 |
| HR | Nbs1 |
| HR | H2AX |
| HR | Hop2 |
| HR | PALB2/FANCN |
| HR | PCNA |
| HR, FA | RAD51 (FANCR) |
| HR | RAD51AP1 |
| HR | Rad51B |
| HR, FA | Rad51C (FANCO) |
| HR | Rad51D |
| HR, SSA | RAD52 |
| HR | RAD54 |
| HR | XRCC2 |
| HR | XRCC3 |
| HR | RAP80 |
| HR | RMI1+ |
| HR | RMI2+ |
| HR | RNF168 |
| HR | RNF8 |
| HR | RA1A |
| HR | RPA2 |
| HR | RPA3 |
| HR | GIY |
| HR | GIY-YIG |
| HR | SLX1 |
| HR | SLX4 (FANCP) |
| HR | SMC1 |
| HR | SMC3 |
| HR | SPO11 |
| HR | TIP60 |
| HR | TOPO II |
| HR | TOPOIII |
| HR | UBC13 |
| HR | WRN |
| HR | ChK1 |
| HR | ChK2 |
| HR | p53 |
| HR | CDC25 |
| HR, MMEJ, SSA | Srs2 |
| HR, MMEJ, SSA, NER | Xpf |
| HR, MMEJ | Pol δ (delta), Pol32 |
| HR | POLD1 |
| HR | POLD2 |
| HR | POLD3 |
| HR | POLD4 |
| HR | Pol ξ |
| HR, MMEJ, BER, NER, SSA | Ligase I |
| HR, MMEJ, BER, NER | Ligase III |
| MMEJ | Pol θ (theta) |
| MMEJ | Histone H1 |
| MMEJ | WRN |
| MMEJ, NHEJ | Pol β (beta), POLB |
| MMEJ, NHEJ | Pol4 |
| MMEJ, TLS | Pol η |
| MMEJ, TLS, HR | Pol ξ |
| MMEJ | PNK |
| SSA | RAD59 |
| SSA | RPA |
| SSA | XRS2 |
| SSA | Msh2 |
| SSA | Msh3 |
| SSA | Rad10 |
| SSA | DNA2 |
| SSA | RFC, RFC-like |
| SSA | PCNA-like protein |
| | (Rad1, Hus1, Rad9) |
| FA | FANCA |
| FA | FANCB |
| FA | FANCC |
| FA | FANCE |
| FA | FANCF |
| FA | FANCG |
| FA | FANCI |
| FA | FANCJ (BRIP1) |
| FA | FANCL |
| FA | FANCN |
| FA | FANCP |
| FA | FANCT |
| FA | FANCM |
| FA | FAAP100 |
| FA | FAAP24 |
| FA | FAAP20 |
| FA | FAAP16 |
| FA | FAAP10 |
| FA | BOD1L |
| FA | UHRF1 |
| FA | USP1 |
| FA | UAF1 |
| FA | AN1 |

Examples of NHEJ inhibitors (=inhibitors of PARP1, Ku70/80, DNA-PKcs, XRCC4/XLF, Ligase IV, Ligase III, XRCCI, Artemis, PNK) include without limitation, NU7441 (Leahy et al., Identification of a highly potent and selective DNA-dependent protein kinase (DNA-PK) inhibitor (NU7441) by screening of chromenone libraries. (Bioorg. Med. Chem. Lett. (2004) 14:6083-6087), NU7026 (Willmore et al. A novel DNA-dependent protein kinase inhibitor, NU7026, potentiates the cytotoxicity of topoisomerase II poisons used in the treatment of leukemia. (Blood (2004) 103), Olaparib, DNA Ligase IV inhibitor, Scr7 (Maruyama et al., Increasing the efficiency of precise genome editing with CRISPR-Cas9 by inhibition of nonhomologous end joining. (Nat. Biotechnol. (2015) 33:538-542), KU-0060648 (Robert et al., Pharmacological inhibition of DNA-PK stimulates Cas9-mediated genome editing. Genome Med (2015) 7:93), anti-EGFR-antibody C225 (Cetuximab) (Dittmann et al., Inhibition of radiation-induced EGFR nuclear import by C225 (Cetuximab) suppresses DNA-PK activit.” Radiother and Oncol (2005) 76: 157), Compound 401 (2-(4-Morpholinyl)-4H-pyrimido[2,l-a]isoquinolin-4-one), Vanillin, Wortmannin, DMNB, IC87361, LY294002, OK-1035, CO 15, NK314, PI 103 hydrochloride, to name just a few exemplary inhibitors.

MMEJ inhibitors, include, but are not limited to, MRE11 inhibitors such as Mirin and derivatives (Shibata et al, Molec. Cell (2014) 53:7-18), inhibitors of PolQ, inhibitors of CtIP. See Sfeir and Symington, “Microhomology-Mediated End Joining: A Back-up Survival Mechanism or Dedicated Pathway?” Trends Biochem Sci (2015) 40:701-714).

Examples of HR inhibitors include, but are not limited to RI-1 and B02.

Examples of HR stimulators include, but are not limited to, RS-1 (RAD51 stimulator).

NHEJ stimulators, include, but are not limited to, IP6 (Inositol Hexakisphosphate, DNA-PK enhancer, Hanakahi 2000, Ma 2002, Cheung 2008).

A downmodulation of a DRP reduces the activity of such a DRP in a cell or population of cells. A downmodulation of a DRP can be by 10%, 20%, 30%, 40%, 50%, 60%, 70%, 80%, 90%, 95% or 100% of the repair activity (hereinafter “activity”) without the downmodulation. The downmodulation can be achieved in many ways, such as, but not limited to, contacting said cell or population of cells, with one or more inhibitor(s), such as a chemical inhibitor of the DRP/a component thereof, inactivating the DRP/a component thereof, downregulating the DRP/a component thereof (e.g. by contacting or expressing in said cell or population of cells one or more inhibitory nucleic acids such as a miRNA, a siRNA, a shRNA or any combination thereof) and/or mutating one or more genes of said DRP/a component thereof.

In a preferred embodiment a DRP is downmodulated that is either non-productive or competes with another DRP and is thus referred to as a competing pathway or non-productive pathway.

For example, a NHEJ pathway may be inhibited to favor productive integration of an exogenous DNA by e.g. MMEJ and related mechanisms. In the context of the present invention any active DRP may compete with another active DRP in a cell and is thus a competing DR pathway. A non-productive DRP in the context of the present invention is a pathway that will not or will only inefficiently mediate the integration of exogenous DNA into the cell genome. For example, synthesis-dependent strand annealing (SDSA), Break-induced replication (BIR), base excision repair (BER), nucleotide excision repair (NER), mismatch repair (MMR), DNA damage response (DDR), Blunt End Joining, single strand break repair (SSBR), and interstrand crosslink repair (ICL) are generally inefficient in mediating the integration of exogenous DNA.

The downmodulation of one DRP generally results in one or more other DNA repair pathways to take over the repair work of the downmodulated DRP. The one or more DRPs that take on the repair work is generally upmodulated. An upmodulation of the one or more DRPs can be by 10%, 20%, 30%, 40%, 50%, 60%, 70%, 80%, 90%, 95% or 100% of the activity without the downmodulation. A DRP that is upmodulated as a result of downmodulation of another competing DRP is considered “favored” (or enhanced) relative to the downmodulated DRP. The degree of favoring/enhancing may be proportional to the degree of downmodulation and may, e.g., be a 10%, 20%, 30%, 40%, 50%, 60%, 70%, 80%, 90% or 95% higher activity relative to the activity without the downmodulation of the downmodulated DRP. The activity of the downmodulated DRP may shift to one pathway, but may also shift to two or more pathways that take over the DNA repair functions of the downmodulated DRP. Apart from downmodulating another DRP, a DRP may also be upmodulated, by, e.g., expressing, including causing the overexpression of, one or more components of said DRP in said cell or population of cells, introducing into said cell or population of cells, the component of the said DRP heterologously, contacting said cell or population of cells, with one or more modulator, preferably a stimulator, such as a chemical stimulator of the one or more component of the said DRP, mutating one or more genes of said DRP, wherein said mutating enhances expression or activity of the one or more components of the said DRP.

In a preferred embodiment, cells are synchronized in a cell cycle phase, such as the G1, S or G2 phase, by the physical addition of a modulator of the cell cycle prior to transfection. Cell synchronization in G1 phase supports higher viability and cell recovery during antibiotic selection.

Moreover, the DNA double-strand breaks reparation pathways by end-resection were described to be at their optimal activity during phases S and G2 of the cell cycle. Previous work suggested that stable transgene integration in CHO cells was favored by microhomology-mediated end joining (MMEJ), single strand annealing (SSA) or homologous recombination (HR) mechanisms (Grandjean et al. (2011), High-level transgene expression by homologous recombination-mediated gene transfer.” Nucl. Acids Res., 39, e104; Kostyrko et al. (2017), “MAR-Mediated transgene integration into permissive chromatin and increased expression by recombination pathway engineering,” Biotechnol. Bioeng., 114, 384-396).

A nucleic acid having substantial identity with another nucleic acid is part of the present invention. A nucleic acid has substantial identity with another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases.

Identity means the degree of sequence relatedness between two polynucleotides sequences as determined by the identity of the match between two strings of such sequences, such as the full and complete sequence. Identity can be readily calculated. While there exists a number of methods to measure identity between two polynucleotide sequences, the term “identity” is well known to skilled artisans (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity between two sequences include, but are not limited to those disclosed in Guide to Huge Computers, Martin J. Bishop, ed., Academic Press, San Diego, 1994, and Carillo, H., and Lipman, D., SIAM J Applied Math. 48: 1073 (1988). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, J., et al., Nucleic Acids Research 12(1). 387 (1984)), BLASTP, BLASTN, FASTA (Altschul et al. (1990); Altschul et al. (1997)). The well-known Smith Waterman algorithm may also be used to determine identity.

As an illustration, by a nucleic acid having a nucleotide sequence having at least, for example, 95% “identity” to a reference nucleotide sequence means that the nucleotide sequence of the nucleic acid is identical to the reference sequence except that the nucleic acid sequence may include up to five point mutations per each 100 nucleotides of the reference nucleotide sequence. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

### EXAMPLES

### Example 1: CHO Cell Synchronization Increases Stable Integration of Recombinant Protein Transgene and Transfectability

The example demonstrates that stable transgene integration events were increased after cell synchronization in phase S and G2 prior to transfection. Moreover, this indicates that cell synchronization was shown to be a method to support a higher cell viability and to achieve increased recovery of cells during antibiotic selection relative to non-synchronized cells.

In the first part of this study, the aim was to determine the cell cycle phases of CHO cell line.

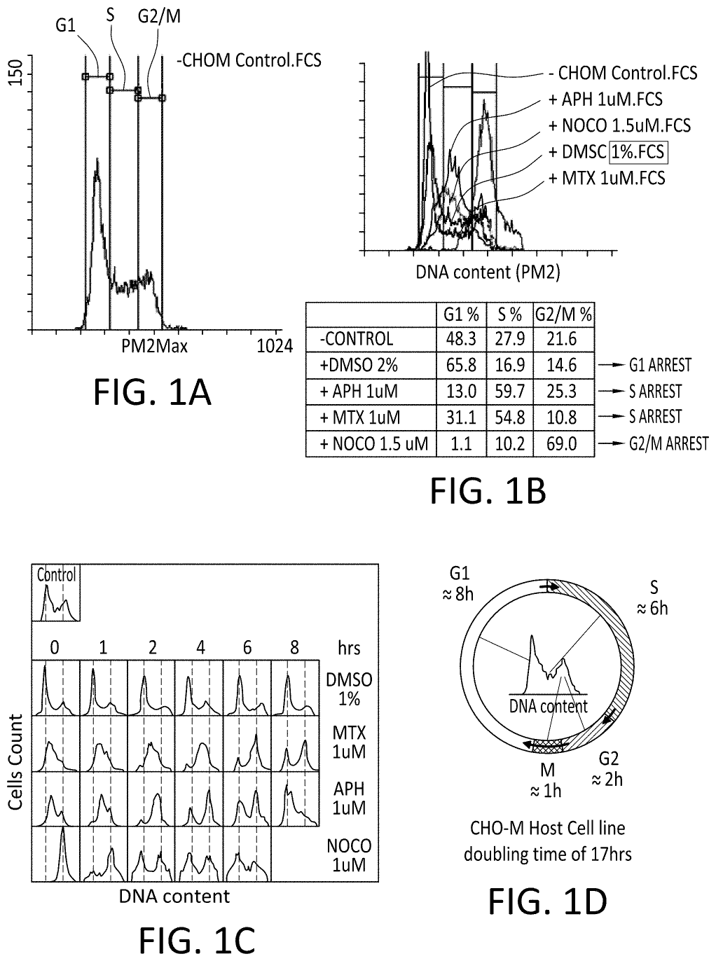

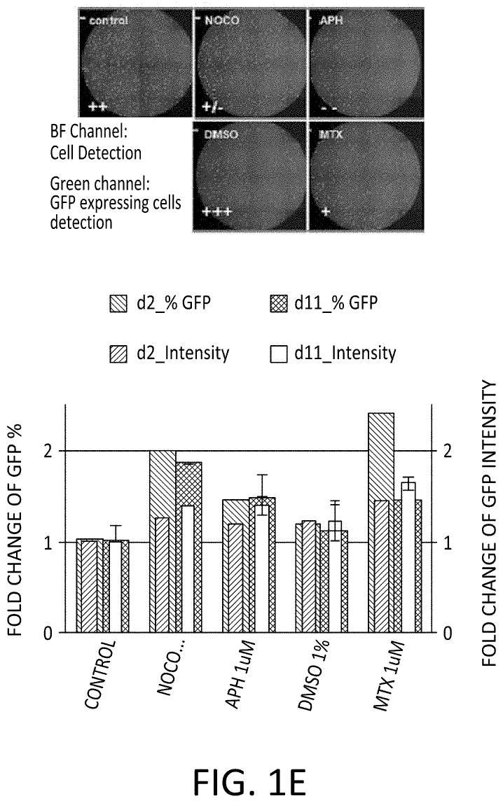

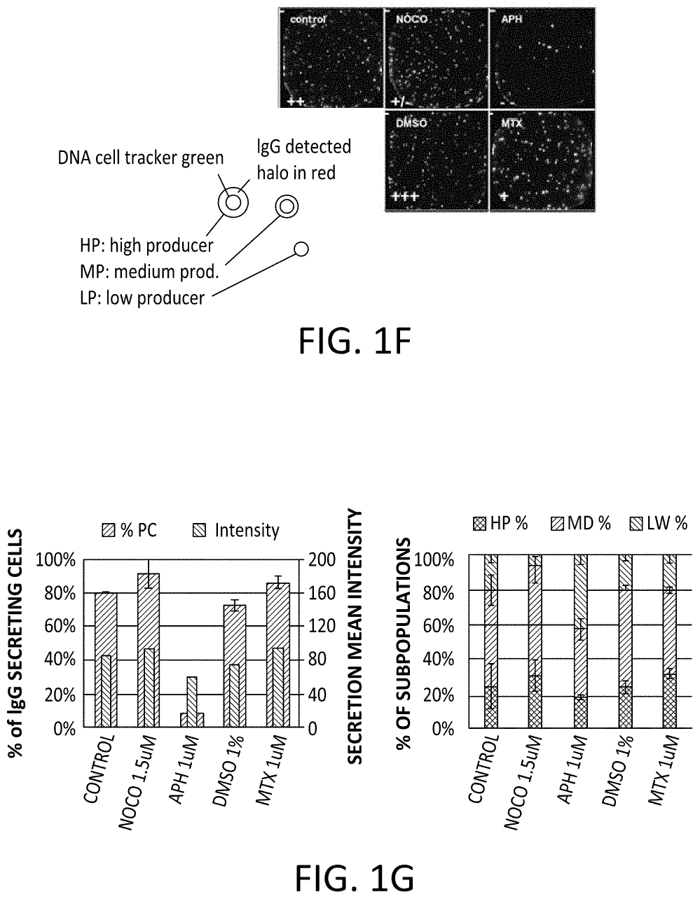

SURE CHO-M cell™ line (SELEXIS SA, Switzerland, see: U.S. Pat. Nos. 7,129,062, 8,252,917 and 9,879,297, and U.S. Patent Applications No. 20110061117 and 20120231449, the disclosures of which are incorporated herein by reference in their entirety) were cultivated overnight in asynchronous condition (FIG. 1A) or in presence of DMSO 1%, aphidicholin (APH) 1 μM, methotrexate (MTX) 1 uM or nocodazole (NOCO) 1.5 μM (FIG. 1B). Cells were then fixed in ethanol and labelled overnight at 4° C. with propidium iodide (PI; 50 μg/ml, Sigma) in presence of RNAse (0.5 μg/ml, Sigma). DNA content index histograms were acquired on Guava EasyCyte System® using INCYTE acquisition software. Each phase of cell cycle was defined according to the drug treatment as G1 arrest (DMSO); S phase (APH); G2 (NOCO). The % of G0/G1, % of S and % of G2/M arrested cells were then calculated (see Table FIG. 1B).

Cells were synchronized overnight with DMSO 1%, aphidicolin (APH) 1 μM, methotrexate (MTX) 1 μM or nocodazole (NOCO) 1.5 μM provided by Sigma (FIG. 1C). After 18 hrs of incubation cells were centrifuged, rinsed twice into PBS 1× and resuspended in complete culture medium. Cell cycle was analyzed by flow cytometry at release (18 h with drug) and at 1, 2, 4, 6 and 8 h after release. Released cells were then further cultivated according to the indicated time points. Asynchronous (control) cells were included as controls. DNA content index histograms were acquired on Guava EasyCyte System® (MILLIPORE) using INCYTE acquisition software. Scheme of the CHO-M cell cycle phase duration is shown in FIG. 1D.

As shown in FIG. 1A and FIG. 1B, DNA content of asynchronous cell show a distribution of 48% of G0/G1, 28% of S and 22% of G2/M. According to the cell cycle progression of synchronized CHO cells after drugs released, the duration of each cell cycle phase was determined. CHO cell line demonstrated a doubling time of 17 h with a G0/G1 phase of 8 h, a S phase of 6 h, a G2/M of 3 h (phase M of 1 h).

In the second part of this study, the aim was to determine the effects of CHO cell synchronization on the efficiency of stable transgene integration and cell recovery during antibiotic selection.

Asynchronous and synchronized SURE CHO-M cell™ lines (SELEXIS SA, Switzerland) were transfected with eGFP- (FIG. 1E) or Trastuzumab IgG-expression SLX-vectors (FIG. 1F and FIG. 1G) immediately after drugs release. Transfection was done by microporation (NEON TRANSFECTION SYSTEM, INVITROGEN), generating a heterogeneous pool of transfected cells. 24 h after transfection, 5 ug/ml puromycin selection agent (GIBCO) was added to the BalanCD medium (IRVINE) supplemented with 6 mM L-Glutamine (HYCLONE). Expression pools performances were then evaluated for GFP or IgG at different time points after transfection. The percentage and GFP fluorescence mean intensity (FMI) level were evaluated on cytometer imager at d2 and d11 post-transfection (CELIGO S; NEXCELOM). Results were represented as fold change of control cells (FIG. 1E; histograms).

The percentage and secretion mean intensity (SMI) of IgG-producing cells were determined at d2 (FIG. 1F) and d10 (FIG. 1G) post-transfection using Cell Secretion Assay methods (CSA). Briefly, cells were incubated at 37° C. overnight, with a green-fluorescent cell detection reagent (CellTracker™ Green CMFDA Dye) and with an anti-human IgG PE-conjugated antibody. After overnight incubation, culture plates were imaged using CELIGO Cell Cytometer (NEXCELOM). The anti-human IgG PE-conjugated antibody interacted with the secreted recombinant IgG by forming fluorescent detectable secretion network closer to the single cell—the halo of secretion (PC=producing cells, HP=high producers, MD=medium producers and LW=low producers, see, e.g. FIG. 1G).

As shown in FIG. 1E, the analysis of GFP-expressing cells day 2 and day 11 after transfection demonstrated that the percentage and mean intensity of expressing cells were higher for cells synchronized in phase S or G2 prior to transfection than those of non-synchronized cells (NOCO and MTX pictures and histograms compared to Control).

As shown in FIG. 1F and FIG. 1G, the analysis of Trastuzumab-expressing cells 2 days post-transfection demonstrated that the % and mean intensity of expressing cells were higher for cells synchronized in phase S or G2 compared to asynchronized cells (NOCO and MTX pictures and histograms compared to Control).

After 10 days of antibiotic selection, stable trastuzumab-IgG cells were re-analyzed (FIG. 1G; histograms). Compared to control cells, the percentage and mean intensity of IgG-expressing cells were slightly higher for cells synchronized in phase S or G2 (FIG. 1F; % PC histograms). Nonetheless, the analysis of low, medium and high producing cells distribution in the different pools, shown a significant increase of high-producing cells subpopulations of synchronized cells in phase S and G2 (FIG. 1G; % HP histograms).

In sum, these experiments suggested that stable transgene integration events were increased after cell synchronization in phase S and G2 prior to transfection. However, synchronization in G1 phase supported a higher cell viability and recovery of cells during antibiotic selection.

Moreover, the DNA double-strand breaks reparation pathways by end-resection were described to be at their optimal activity during phases S and G2 of the cell cycle. These data suggested that stable transgene integration in CHO cells was favored by microhomology-mediated end joining (MMEJ), single strand annealing (SSA) or homologous recombination (HR) mechanisms.

### Example 2: Addition of Specific Enzymes During Transfection Indirectly Increases Pool Antibody Productivity and Antibody Productivity in CHO Cells

Surprisingly, we discovered that specific enzymes, such as the PvuI restriction enzyme, targeting determined digestion patterns, methylation sensitivity and different number of potential sites within genome, can be used to indirectly improve the antibody productivity of CHO cells.

Thus, different enzymes such as restriction enzymes, were tested according to their different digestion patterns (e.g., size of recognition pattern, composition of recognition pattern, type and cut pattern) as well as different sensitivity to methylation and different number of potential sites within CHO genome (Table 2). The aim was to determine if they could direct transgene facilitated insertion, or increase the number of transgene inserted or stability of transgene, and, indirectly, affect productivity.

0.34 million of SURE CHO-M cells™ (SELEXIS SA, Switzerland) were transfected with 3 ug of different antibody DNA fragments, respectively Trastuzumab (Tras) or Adalimumab (ADA), supplemented with 6 units of different enzymes, PvuI, SbfI, AscI or BstBI (NEB). Transfection was done by microporation (Neon Transfection system®, INVITROGEN), generating a heterogeneous pool of transfected cells. 24 h after transfection, 5 ug/ml puromycin selection agent (GIBCO) was added to the BalanCD medium (IRVINE) supplemented with 6 mM L-Glutamine (HYCLONE). Growth and performance of the expanded pools were evaluated in spin tube in a 9-day fedbatch process using Acto CHO A+B feed® (GE HEALTHCARE). Fed-batcg cultures were initiated at cell concentrations of 0.3×106 cells/ml in 5 mL working volume run. Cell density and cell viability along the process were evaluated by using a Guava System® (MILLIPORE) and supernatant sample was collected. Antibody product titer was evaluated by ELISA capture assay against the collected supernatant, at day 9 of the fedbatch process. Productivity per cell per day (PCD) was calculated as function of titer and viable cell density during the fedbatch process.

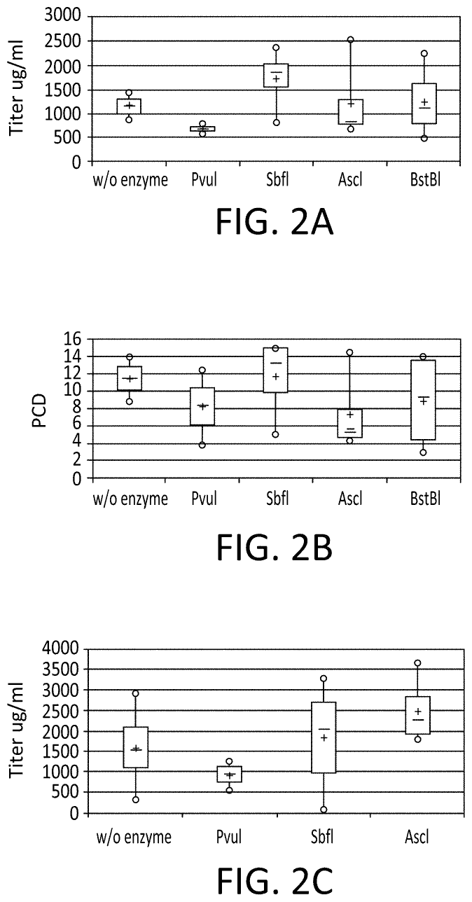

As shown in FIG. 2A and FIG. 2B, SbfI and BstBI and, to a lesser extent, AscI, show indirect effects to increase cell pool productivity, enhancing both the immunoglobulin titer and the specific cell productivity, as compared to non-enzymatic treated cells. Interestingly, these two graphs show that the addition of specific enzymes such as restriction enzymes during transfection indirectly increase pool antibody productivity in CHO cells.

In the second part of this study, different enzymes, including restriction enzymes, were tested according to their different digestion patterns (e.g., size of recognition pattern, composition of recognition pattern, type and cut pattern) as well as different sensitivity to methylation and different number of potential sites within CHO genome (Table 2). The aim was to determine if there were direct effects on transgene facilitated insertion, number of transgenes inserted, stability of transgene and indirectly, effects on productivity. But in that experiment, the aim was also to determine effects at the clone level in order to avoid that pool heterogeneity could mask some effects. Therefore, corresponding results were expected to be more distinct.

SURE CHO-M cells™ (SELEXIS SA, Switzerland) were transfected with 3 ug of different of Adalimumab (ADA), supplemented with 6 units of different enzymes, PvuI, SbfI, AscI or BstBI (NEB). Selected cell pools were then plated in semi-solid medium (CLONEMEDIA, Molecular Device) and plates were incubated at 37° C. with 5% CO2, in a humidified incubator in order to isolate single cell colonies. Expanded colonies were picked using ClonePix™ FL Imager (MOLECULAR DEVICE) and transferred to 96-well plates. Clones were then successively ranked by ELISA titration assay and expanded. Growth and performance of the 6 top clones of each enzymatic condition were evaluated in spin tube in a 9-day fedbatch process using Acto CHO A+B feed (GE HEALTHCARE). Fed-batch cultures were initiated at cell concentrations of 0.3×10⁶ cells/ml in 5 mL working volume run. Cell density and cell viability along the process were evaluated by using a Guava System (MILLIPORE) and supernatant sample was collected. Antibody product titer was evaluated by ELISA capture assay against the collected supernatant, at day 9 of the fedbatch process. Productivity per cell per day (PCD) was calculated as function of titer and viable cell density during the fedbatch process.

As shown in FIG. 2C, SbfI and AscI clearly mediate increased clone productivity (titer) as compared to non-enzymatic treated cells. Interestingly, this graph shows that the addition of specific enzymes such as restriction enzymes SbfI or AscI during transfection indirectly increase antibody productivity in CHO cells.

Overall, an increase rate of pool antibody productivity and antibody productivity could be observed after adding selected restriction enzymes such as SbfI and AscI during the transfection of CHO cells.

### Example 3: Modulation of DNA Repair Pathways Promotes Better Transgene Insertion Resulting in Productivity Increase in CHO Cells

Here, an increase rate of productivity of CHO cells, by modulating DNA repair pathways, resulting in favoring one or more said pathways could be demonstrated.

The aim of this study was to inhibit the nonhomologous end-joining repair pathway in view of promoting alternative repair pathways to boost transgene integration and resulting in an indirect increased productivity of CHO cells modified in a such way.

SURE CHO-M cells™ (SELEXIS SA, Switzerland) were treated with 0.4 μM of DNA-PK inhibitor Nu7441 (TOCRIS) just before transfection of 3 μg of the antibody DNA fragment, respectively Trastuzumab (Tras) or Adalimumab (ADA). Transfection was done by microporation (Neon Transfection system, Invitrogen). 24 h after transfection, 5 μg/ml puromycin selection agent (Gibco) was added.

Growth and performance of the 6 top clones of each enzymatic condition were evaluated in spin tube in a 9-day fedbatch process using Acto CHO A+B feed (GE Healthcare). Fed-batch cultures were initiated at cell concentrations of 0.3×10⁶ cells/ml in 5 mL working volume run. Cell density and cell viability along the process were evaluated by using a Guava System (Millipore) and supernatant sample was collected. Antibody product titer was evaluated by ELISA capture assay against the collected supernatant, at day 9 of the fedbatch process. Productivity per cell per day (PCD) was calculated as function of titer and viable cell density during the fedbatch process.

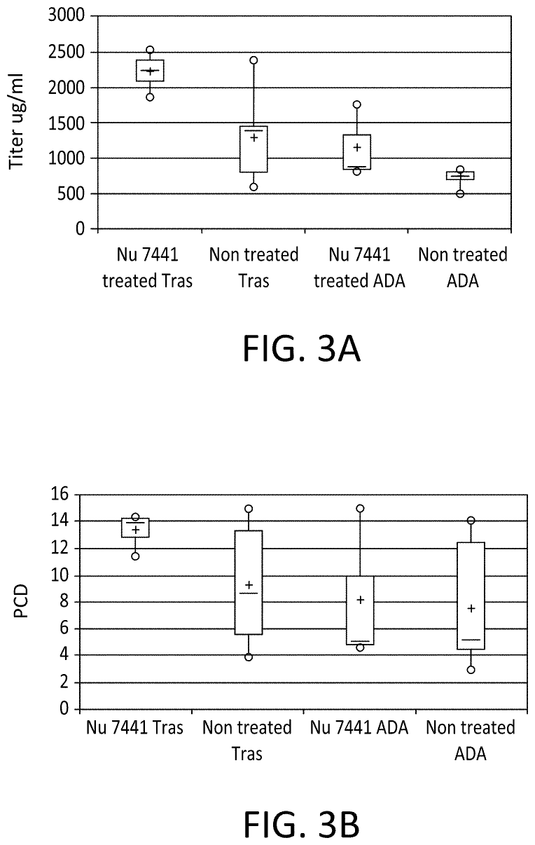

As shown in FIG. 3A and FIG. 3B, for both antibody molecules, treatment of CHO cells with Nu7441 showed an increase productivity. This is also correlated with a clear increased PCD for Trastuzumab while PCD effect remains less evident for adalimumab.

By blocking the non-homologous end-joining repair pathway (NHEJ), the DNA-PK inhibitor Nu7441 may have indirectly enhanced alternative DNA repair pathways such as homology-directed repair (HDR) pathway, and thus promoted better transgene insertion, resulting in productivity increase in CHO.

### Example 4: CHO Cell Synchronization Combined to CRISPR/Cas-Mediated Transgene Integration Increases of Productivity of Recombinant Protein Expression

The experiments of this example demonstrate that combining the CHO cell synchronization in a defined cell phase to a transgene integration, performed as in the previous examples, leads to an increase of productivity of recombinant protein expression by such modified cells.

### Impact of Cell Synchronization on the Transgene Integration Using CRISPR/Cas Targeting Expression System

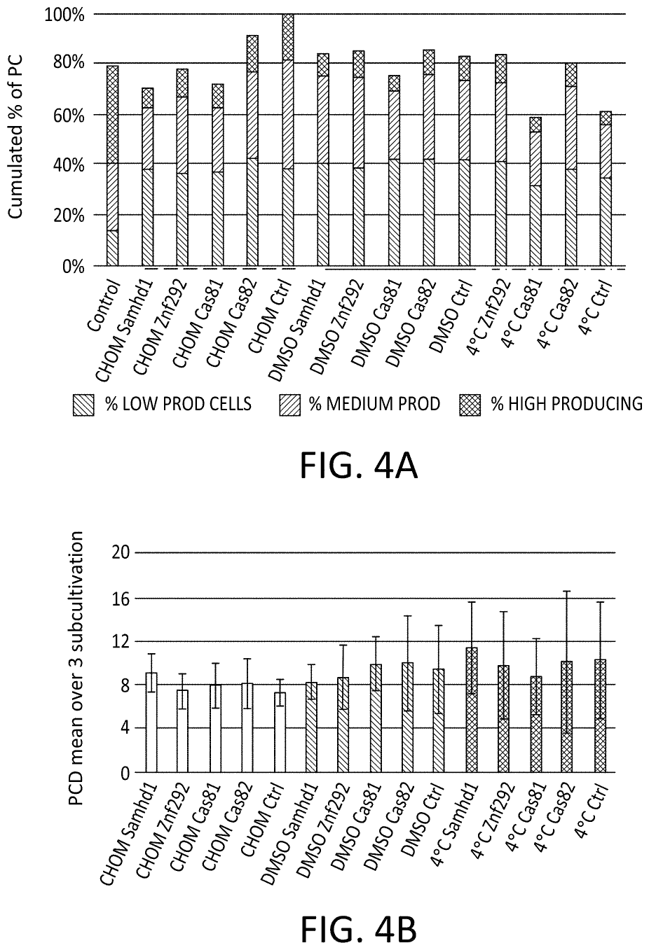

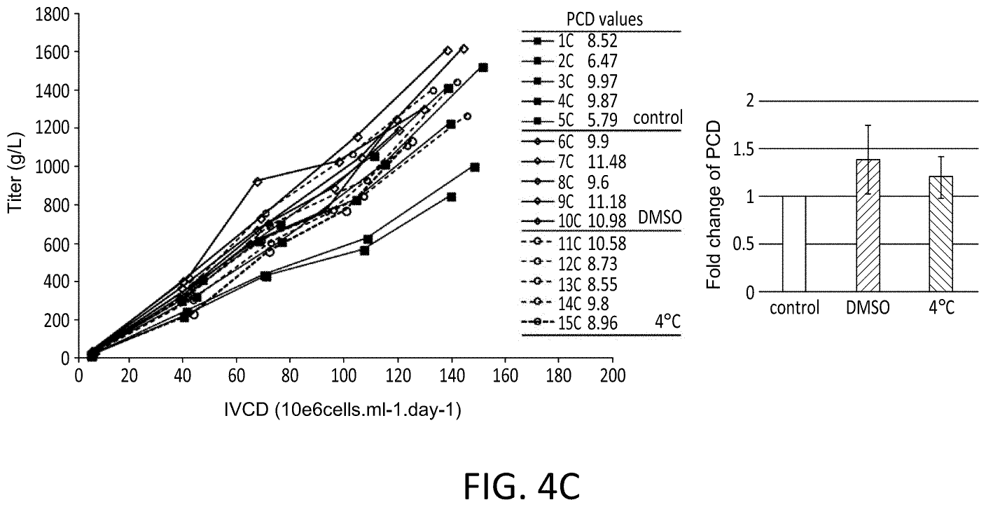

SURE CHO-M cells™ (SELEXIS SA, Switzerland) were synchronized overnight with DMSO 1% or incubation at 4° C. After 18 hrs of incubation, cells were centrifuged, rinse twice into PBS 1× and resuspended in complete culture medium. Asynchronous and synchronized cells were transfected with IgG-trastuzumab expressing vectors and cultivated under antibiotic selection for 10 days. A Cell Secretion Assay (CSA) was performed to determine the % of producing cells (FIG. 4A). The histogram showed the % the high-, medium- and low-producing subpopulations (indicated as HP, MP and LP, respectively).

Stable expressing pools were subcultivated in complete culture medium for 4 subsequent passages in spin tubes (5 ml wv) (FIG. 4B). Cell density (Cv·ml⁻¹) and IgG titer values (μg·ml⁻¹) were determined. Histograms showed the specific productivity (pg·cell⁻¹·day⁻¹) as mean values of 4 cultivation passages. Growth and performance of each stable established IgG-expressing pool was then evaluated in spin tube in a 9-day fedbatch process using Acto CHO A+B feed (GE Healthcare). Fed-batch cultures were initiated at cell concentrations of 0.3×10⁶ cells/ml in 5 mL working volume run. (FIG. 4C). The specific IgG productivity was determined as the slope of IgG concentration versus the integral number of viable cells (IVCD) and expressed as pg per cell and per day (pcd). Histograms represented the fold change of productivity per cell per day (PCD) obtained for DMSO and 4° C. pre-treatment compared to their respective untreated-controls cells.

As shown in FIG. 4A, as plasmid-expression vector, CRISPR/CAS9 targeting expression system leads to similar proportion of high producing CHO cells by CSA analysis. As shown in FIG. 4B, the analysis of cells productivity through 4 batch cultivation and fed-batch production run, shows comparable results for various IgG-trastuzumab expression system compared to their counterpart control cells. As shown in FIG. 4C (histogram), this clearly illustrates that CHO cells synchronization in G1 phase using DMSO or 4° C. pre-cultivation condition leads to a significant increase of productivity for plasmid-based expression system as well as for CRISPR/Cas9-mediated expression system.

In sum, these experiments suggested the percentage of producing cells is increased by carrying out transgene integration based on a CRISPR-Cas system as well as a plasmid-expression vector, and that cell synchronization in G1 phase leads to a significant increase of productivity for plasmid-based expression system as well as for CRISPR/Cas-mediated expression system.

### Example 5: CHO Cell Synchronization Combined to Modulation of DNA Repair Pathways Promote Better Transgene Integration

The experiments of this example demonstrated that combining the CHO cell synchronization in a defined cell phase to modulation of defined DNA repair pathways leads to high cell recovery during antibiotic selection leading to enrichment of high producing cells, favoring a better transgene integration.

Another aim was to evaluate if DNA DSBs repair pathways inhibitor potency may favor recombinant transgene integration in CHO cells in combination with cell cycle synchronization.

SURE CHO-M cells™ (SELEXIS SA, Switzerland) were synchronized overnight with DMSO 1%, aphidicolin (APH) 1 μM, methotrexate (MTX) 1 μM or nocodazole (NOCO) 1.5 μM. After 18 hrs of incubation, cells were centrifuged, rinse twice into PBS 1× and resuspended in complete culture medium. Asynchronous and synchronized cells were transfected with trastuzumab IgG-plasmid expressing vector. Freshly transfected cells were then immediately resuspended and incubated overnight in presence of NU7441, RI-1, RS-1 or Olaparib small molecules (drugs provided by TOCRIS, CALBIOCHEM or APEXBIO TECHNOLOGY) before to change medium and start antibiotic selection.

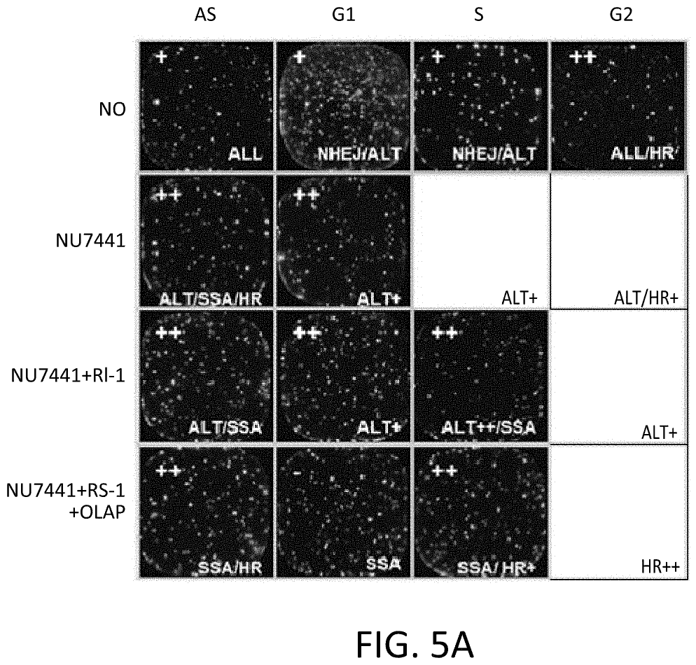

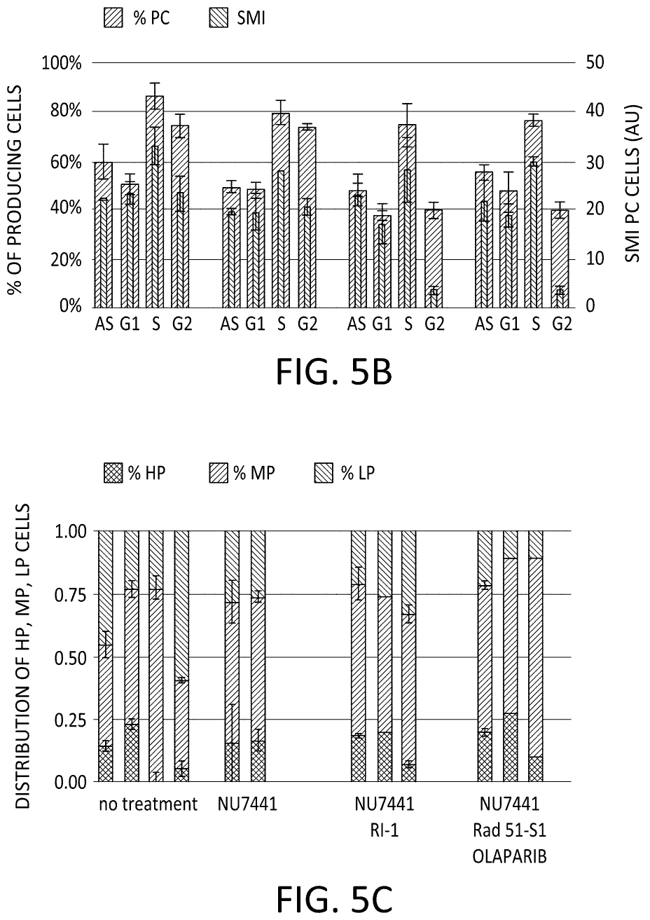

Two days after transfection a cell secretion assay (CSA) was performed to determine the percentage of producing cells (FIG. 5A). Briefly, cells were incubated at 37° C. overnight, with a green-fluorescent cell detection reagent (CellTracker™ Green CMFDA Dye) and with an anti-human IgG PE-conjugated antibody. After overnight incubation, culture plates were imaged using CELIGO Cell Cytometer (Nexcelom). The anti-human IgG PE-conjugated antibody interacted with the secreted recombinant IgG by forming fluorescent detectable secretion network closer to the single cell—the halo of secretion. Cell recovery behavior during antibiotic selection was monitored for each transfection condition and recorded as + or − signs. Two days (FIG. 5B) and ten days (FIG. 5C) after selection, stable IgG-expressing cells were re-analyzed by CSA. The histograms show the percentage and secretion mean intensity (SMI) of total producing cells as well as the high-, medium- and low-producing subpopulations.

The potency of different DNA DSBs repair pathways inhibitors to favor recombinant transgene integration was assessed in combination with CHO cell cycle synchronization. NU7441, is a DNA-dependent protein kinase (DNA-PK) inhibitor. DNA-PK in combination with Ku70/80 is important for successful DNA DSBs repair by NHEJ mechanism. RI-1 is a small molecule inhibitor of RAD51 protein. The inhibition of RAD51 protein leads to inhibition of DNA DSBs repair by homologous mechanism (HR). Moreover, it was previously described that RI-1 stimulates the single-strand annealing mechanism of DSBs repair (SSA). SSA does not involve the proteins of the NHEJ nor of the HR pathway. RS-1 is a homologous recombination enhancer. Olaparib is a potent inhibitor of poly(ADP-ribose) 22polymerase PARP1. This later is involved in association with Pole polymerase in the DNA DSBs repair mechanisms referred as error-prone alternative end joining (alt-EJ) or microhomology-mediated end-joining (MMEJ). NHEJ-mediated DNA DSBs repair mechanisms is not to be cell cycle regulated. Contrary, HR mechanisms are described to be restricted to S/G2 phases using the MRN complex consisting of MRE11A, RAD50 and NBN (NBS1) proteins. As well, MMEJ, Alt-EJ and SSA end-resected repair were active during S and early G2 phases when the sister chromatid is not available to favor homologous recombination.

Freshly transfected cells were incubated in presence of various NHEJ, HR, MMEJ or SSA modulators immediately after electroporation to identify the DNA repair pathway involved in transgene integration and depending on the targeted cell cycle phase.

The percentage and SMI of trastuzumab-IgG producing cells was determined 2 days after transfection using CSA. These analyses demonstrated that at early evaluation the best transfectability and IgG-expression level were obtained with CHO-M cell synchronized in phases S independently of drug treatment applied to inhibit DNA repair mechanisms (FIG. 5A and FIG. 5B). G2-synchronized cells with or without NHEJ inhibition, exhibited early significant higher production performance compared to asynchronous cells. Moreover, G2-synchronized cells demonstrated a strong sensitivity to MMEJ and HR inhibition treatment. Together these results confirmed the prevalence of HR and MMEJ in repairing DNA DSBs during phases S/G2.

Independently of DNA DSBs pathways inhibition, it was shown that S- and G2-synchronized cells failed to pass the antibiotic selection, suggesting a lethal cytotoxic effect of methotrexate and nocodazole treatment and DNA repair pathways inhibition.

After 10 days of antibiotic selection, the analysis of stable IgG-producing cells obtained after transfection of asynchronous and DMSO-treated cells with or without DNA repair modulators treatment demonstrated an increase of high-producing subpopulation for G1-synchronized cells compared to their respective asynchronous controls (FIG. 5C, % HP).

In sum, the combination of S and G2 arrest and the inhibition of DSB repair pathways by NHEJ (Nu7441) or HR (RI-1; Olaparib) mechanisms demonstrated higher proportion of high-expressing cells immediately after transfection. However, cells drug release did not lead to restored cell cycle progression and DNA repair, but it induced CHO cell death during antibiotic selection. Overall, it suggested that G1- but not S and G2-cell synchronization, and inhibition of NHEJ DNA repair during cell transfection promoted better transgene integration by maintaining high cell recovery during antibiotic selection leading to enrichment of high producing cells.

### Example 6: Combination of Cell Cycle Synchronization and DNA Repair Pathways Modulations Improve Integration by Specific Enzymes

Here it was demonstrated that the combination of cell synchronization to the modulation of DNA repair pathways favor a better recombinant transgene integration in presence of nucleases generating double-strand breaks, resulting in a high degree of recovery during antibiotic selection and enrichment in high producing cells.

The aim of the study was to determine the impact of the combination of cell synchronization in G1 with the inhibition of NHEJ DNA repair mechanism in presence of Sbf1 restriction enzyme on the transgene integration efficiency on CHO cell line.

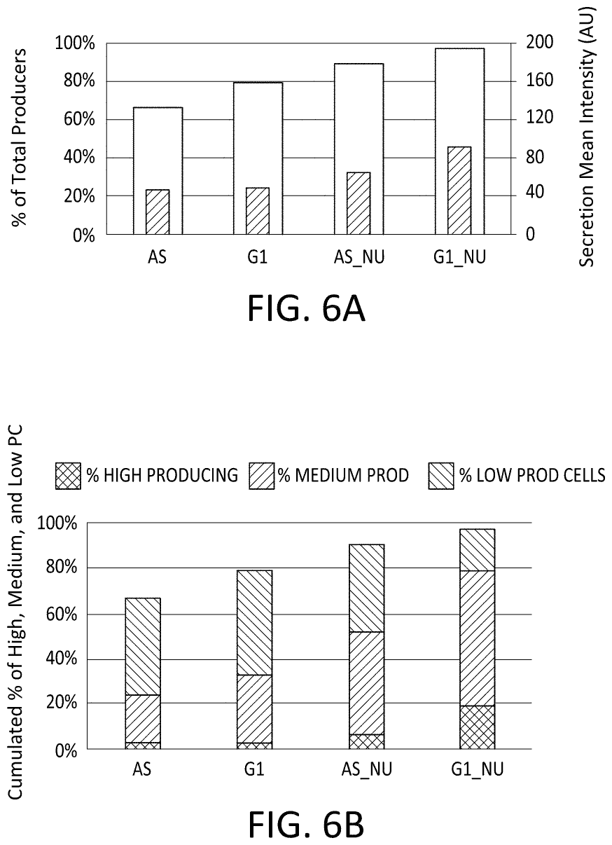

Asynchronized (AS) or G1-synchronized (G1) SURE CHO-M cells' (SELEXIS SA, Switzerland) were transfected with trastuzumab IgG-expressing vector in presence of Sbf1 restriction enzyme. Transfected cells were incubated overnight in presence of the NHEJ inhibitor—NU7441 (0.4 mM)—before change of medium and start antibiotic selection. The histograms show cell secretion assay (CSA) as percentage (white bar) and secretion mean intensity (SMI) (grey bar) of total producing cells (FIG. 6A) or of the high-, medium- and low-producing subpopulations (FIG. 6B) performed on stable trastuzumab-expressing cells.

After 10 days of antibiotic selection, stable trastuzumab-IgG expressing cells were analyzed by CSA. As shown in FIG. 6A, the percentage and mean intensity of IgG-expressing cells were higher for cells treated with Nu7441 (AS_NU and G1_NU histograms) compared to their counterpart cultivation without NHEJ inhibitor (AS and G1 histograms). Moreover, the combination of G1 phase synchronization and Nu7441 treatment exhibited the best proportion of IgG-producing cells (G1_NU compared to AS). As shown in FIG. 6B, the analysis of low, medium and high producing cells distribution in the different pools, showed a significant increase of high- and medium-producing cells subpopulations of G1 phase synchronized- and Nu7441 treated-cells.

Overall, the results suggest that G1 cell synchronization and inhibition of NHEJ DNA repair favored a better recombinant transgene integration in presence of restriction enzyme-mediated DNA DSBs during CHO cells transfection. This transfection condition results in a high degree of recovery during antibiotic selection and enrichment in high producing cells.

### BIBLIOGRAPHY

Agarwal, M. et al. (1998) Scaffold attachment region-mediated enhancement of retroviral vector expression in primary T cells. J Virol.; 72: 3720-3728.
Bell, A. C. and Felsenfeld, G. (1999) Stopped at the border: boundaries and insulators. Curr Opin Genet Dev, 9, 191-198.
Castilla, J. et al. (1998) Engineering passive immunity in transgenic mice secreting virus-neutralizing antibodies in milk. Nat Biotechnol 16, 349-354.
Certo, M. T., et al., Tracking genome engineering outcome at individual DNA breakpoints. Nature methods, 2011. 8(8): p. 671-6.
Cherng, J. Y., et al. (1999) Effect of DNA topology on the transfection efficiency of poly((2-dimethylamino)ethyl methacrylate)-plasmid complexes. Journal of controlled release: official journal of the Controlled Release Society. 60(2-3): p. 343-53.
Haber, J. E. (1999) DNA repair. Gatekeepers of recombination. Nature. 398(6729): p. 665, 667.
Kim, J. M. et al. (2004) Improved recombinant gene expression in CHO cells using matrix attachment regions. J Biotechnol, 107, 95-105.
Mayan-Santos, M. D., et al. (2008) A redundancy of processes that cause replication fork stalling enhances recombination at two distinct sites in yeast rDNA. Molecular microbiology. 69(2): p. 361-75.
Mjelle, R., et al. (2015) Cell cycle regulation of human DNA repair and chromatin remodeling genes. DNA repair. 30: p. 53-67.
Murnane, J. P., Yezzi, M. J. and Young, B. R. (1990) Recombination events during integration of transfected DNA into normal human cells. Nucleic acids research. 18(9): p. 2733-8.
Poli, J., et al. (2016) Mec1, INO80, and the PAF1 complex cooperate to limit transcription replication conflicts through RNAPII removal during replication stress. Genes & development. 30(3): p. 337-54.
Stuchbury, G. and Munch, G. (2010) Optimizing the generation of stable neuronal cell lines via pre-transfection restriction enzyme digestion of plasmid DNA. Cytotechnology. 62(3): p. 189-94.
Wurm, F. M. (2004) Production of recombinant protein therapeutics in cultivated mammalian cells. Nat Biotechnol. 22(11): p. 1393-8.
Zahn-Zabal, M. et al. (2001) Development of stable cell lines for production or regulated expression using matrix attachment regions. J Biotechnol 87, 29-42.

1. A method of introducing at least one alteration into genomic nucleic acid(s) of a cell or a population of cells, the method comprising:
i) conditioning the cell or population of cells to obtain a conditioned cell or population of cells, and/or
ii) introducing into and/or expressing in said cell or population of cells, one or more molecules that introduce DNA double-strand breaks and/or DNA single-strand breaks into said genomic nucleic acid, and/or
iii) modulating one or more DNA Repair Pathways (DRPs) of said cell or population of cells, wherein the genomic nucleic acid(s), upon i), ii) and/or iii), comprise(s) the at least one alteration.

2. The method of claim 1, wherein said at least one alteration is a genomic disruption, such as one or more deletions of one or more endogenous nucleic acid(s) and/or one or more insertions of one or more exogenous nucleic acid(s).

3. The method of claim 2, wherein said cell or population of cells are transfected with the one or more exogenous nucleic acid (s) and the at least one alteration is an insertion of the one or more exogenous nucleic acids into the genomic nucleic acid(s).

4. The method of claim 2, wherein the exogenous nucleic acid, is a nucleic acid, such as an DNA encoding a RNA and/or protein of interest.

5. The method of claim 1, wherein the conditioned cell or population of cells of i) is subjected to ii) and/or iii) or wherein the cell or population of cells of ii) is subjected to iii).

6. The method of claim 1, wherein said conditioning in i) results in a synchronization of growth of cells in said population of cells, and is preferably adapted to increase a number of the at least one alteration.

7. The method of claim 1, wherein said conditioning in i) comprises:
ia) modulation of the cell cycle of the cell or cells of the cell population, preferably a chemical modulation via a small molecule such as a cell cycle modulator including dimethyl sulfoxide, methotrexate, nocodazole, aphidicolin, hydroxyurea, aminopterin, cytosine arabinoside, thymidine, butyrate, butyrate salt, lovastatin, compactin, mevinolin, mimosine, colchicine, colcemid, razoxane, roscovitine, vincristine, cathinone, pantopon, aminopterin, fluorodeoxyuridine, noscapine, blebbistatin, reveromycin A, cytochalasin D, MG132, RO-3306, or combinations thereof; and/or
ib) temperature based modulation of the cell cycle of said cell or population of cells, such as keeping the culturing temperature above and/or below a threshold temperature, such as 37° C. and/or alternating between a culturing temperature of above and/or below the threshold temperature; and/or
ic) nutrition based modulation of the cell cycle of the cell or cells of the cell population of said cell or population of cells including limiting nutrients in a standard culture medium such as one or more amino acids, and/or
id) an optional physical separation of a sub-population of cells from the cell population, such as cytofluorometry, fluorescence-activated cell sorting, elutriation, centrifugal separation, mitotic shake-off and combinations thereof.

8. The method of claim 7, wherein said temperature-based modulation in ib) comprises:
providing a culturing temperature of less than 37° C. and greater than 30° C., or
providing a culturing temperature of about 4° C.

9. The method of claim 7, wherein said alternating in ib) comprises reducing the culturing temperature below the threshold temperature and then increasing the culturing temperature of said cell or population of cells above the threshold temperature or vice versa.

10. The method according to claim 1, wherein subsequent to the conditioning in i), a number of cells in the population of cells are in a cell cycle phase selected from the group of interphase, G0 phase, G0/G1 phase, early G1 phase, G1 phase, late G1 phase, G1/S phase, S phase, G2/M phase, and/or M phase exceeds the number of cells in said phase prior to the conditioning, preferably cells in the G1 phase, cells in the S phase, cells in the G2 phase.

11. The method according to claim 2, wherein said introduction of the one or more exogenous nucleic acids takes place at a time when said cell or a majority of cells of said population are at the G1, S or G2 phase of the cell cycle.

12. The method according to claim 1, wherein said one or more molecules in ii) are protein(s), nucleic acid molecule(s) encoding said protein(s) or combinations thereof.

13. The method according to claim 12, wherein said one or more molecules are one or more transposases, one or more integrases, one or more recombinases, or one or more nucleases or nickases including engineered nucleases or engineered nickases.

14. The method of claim 13, wherein said one or more nucleases or nickases are selected from the group consisting of a homing endonuclease, a restriction enzyme, a zinc-finger nuclease or a zinc-finger nickase, a meganuclease or a meganickase, a transcription activator-like effector nuclease or a transcription activator-like effector nickase, an RNA-guided nuclease or an RNA-guided nickase, a DNA-guided nuclease or a DNA-guided nickase, a megaTAL nuclease, a BurrH-nuclease, a modified or chimeric version or variant thereof, and combinations thereof, in particular a zinc-finger nuclease or a zinc-finger nickase, a transcription activator-like effector nuclease or a transcription activator-like effector nickase, a RNA-guided nuclease or an RNA-guided nickase, wherein the RNA-guided nuclease or an RNA-guided nickase are optionally part of a CRISPR-based system, restriction enzyme and combinations thereof.

15. The method of claim 14, wherein said nuclease:
degrades the 5′-terminated strand of the DNA break, or
degrades the 3′-terminated strand of the DNA break in particular, degrades up to 3 nucleotides at the DNA break, degrades up to until 5 nucleotides at the DNA break, and/or degrades more than 5 nucleotides at the DNA break,
restriction enzyme is:
not sensitive to DNA methylation, or
is sensitive to DNA methylation.

16. The method according to claim 1, wherein said one or more DRPs in iii) is selected from the group consisting of resection, canonical homology directed repair (canonical HDR), homologous recombination (HR), alternative homology directed repair (alt-HDR), double-strand break repair (DSBR), single-strand annealing (SSA), synthesis-dependent strand annealing (SDSA), break-induced replication (BIR), alternative end-joining (alt-EJ), microhomology mediated end-joining (MMEJ), DNA synthesis-dependent microhomology-mediated end-joining (SD-MMEJ), canonical non-homologous end-joining repair (C-NHEJ), alternative non-homologous end joining (A-NHEJ), translesion DNA synthesis repair (TLS), base excision repair (BER), nucleotide excision repair (NER), mismatch repair (MMR), DNA damage responsive (DDR), blunt end joining, single strand break repair (SSBR), interstrand crosslink repair (ICL), Fanconi Anemia (FA) Pathway and combinations thereof.

17. The method of claim 16, wherein said modulation of the one or more DRPs results in favoring a second DRP or a second set of DRPs over a first DRP or first set of DRPs.

18. The method of claim 16 or 17, wherein said modulation of the one or more DRPs comprises the modulation of a component involved in said one or more DRPs, wherein a component is preferably a protein, a protein complex or a nucleic acid molecule encoding the protein or the protein complex and/or is one or more of components set forth in Table 3.

19. The method of claim 16, wherein the modulation of said one or more DRPs comprises a downmodulation of said one or more DRPs in said cell or population of cells.

20. The method of claim 19, wherein the downmodulation comprises:
contacting said cell or population of cells, with one or more inhibitor (s), such as a chemical inhibitor, of the DRP or a component thereof and/or,
inactivating or downregulating the component of the said DRP, and/or,
mutating one or more genes of said DRP for inhibiting expression or activity of the component of the said DRP.

21. The method of claim 20, wherein said inactivating or downregulating comprises contacting or expressing in said cell or population of cells, one or more inhibitory nucleic acids such as a miRNA, a siRNA, a shRNA or any combination thereof.

22. The method of claim 19, wherein said one or more DRPs that are downmodulated are selected from the group consisting of canonical non-homologous end-joining repair (C-NHEJ), alternative non-homologous end joining (A-NHEJ), homologous recombination (HR), alternative end-joining (alt-EJ), microhomology mediated end-joining (MMEJ), DNA synthesis-dependent microhomology-mediated end-joining (SD-MMEJ) and combinations thereof.

23. The method of claim 19, wherein said downmodulation results in an upmodulation of one or more further DRPs.

24. The method of claim 23, wherein the one or more DRPs downmodulated is a non-productive pathway or competes with the one or more further DRPs.

25. The method of claim 24, wherein the downmodulated DRP is NHEJ and the upmodulated DRP is HR or MMEJ.

26. The method of claim 16, wherein the modulation of said one or more DRPs comprises an upmodulation of said one or more DRPs in said cell or population of cells.

27. The method of claim 26, wherein the upmodulation comprises:
iia) expressing, including causing overexpression of, one or more components of said DRP in said cell or population of cells,
iib) introducing into said cell or population of cells, the component of the said DRP heterologously,
iic) contacting said cell or population of cells, with one or more modulator, preferably a stimulator, such as a chemical stimulator of the one or more component of the said DRP,
iid) mutating one or more genes of said DRP, wherein said mutating enhances expression or activity of the one or more component of the said DRP, and optionally a downmodulation according to any one of claims 19-26.

28. The method according to claim 16, wherein one DRP is modulated.

29. The method according to claim 16, wherein two or more DRPs are modulated.

30. A cell or population of cells, including a prokaryotic or eukaryotic cell or population of cells comprising at least one alteration in its genomic nucleic acids(s) and being made by the method of claim 1.

31. The cell or population of cells of claim 30, wherein the eukaryotic cell is a yeast cell, a fungi cell, an algae cell, a plant cell or an animal cell such as a mammalian cell.

32. The cell or population of cells of claim 31, wherein the mammalian cell is a Chinese Hamster Ovary (CHO) cell.

33. The cell or population of cells of claim 31, wherein the mammalian cell is a human cell.

34. A cell or population of cells according to claim 30 comprising an exogenous DNA encoding one of more protein of interest, integrated into the genome following cleavage by the compound introducing a double-strand break or a single-strand break in said cell.

35. The method of claim 34, wherein the protein of interest is expressed at a level that exceeds a level of expression attained without i), ii) and/or iii), preferably at least at a twofold, threefold or fourfold level.

36. A kit comprising:
(i) one or more cell cycle modulators;
(ii) or one or more nucleases or nickases including engineered nucleases or engineered nickases; and/or
(iii) one or more DRP modulators; and
instructions for using one or more of (i) to (iii) to introduce at least one alteration into a genomic nucleic acid(s) of a cell or a population of cells.

37. The kit of claim 36, wherein
the one or more cell cycle modulators are dimethyl sulfoxide, methotrexate, nocodazole, aphidicolin, hydroxyurea, aminopterin, cytosine arabinoside, thymidine, butyrate, butyrate salt, lovastatin, compactin, mevinolin, mimosine, colchicine, colcemid, razoxane, roscovitine, vincristine, cathinone, pantopon, aminopterin, fluorodeoxyuridine, noscapine, blebbistatin, reveromycin A, cytochalasin D, MG132, RO-3306 or combinations thereof;
the one or more nuclease is a CRISPR-based system, TALE nuclease or a restriction enzyme;
the one or more DRP modulators downmodulate and/or upmodulate a DRP, such as chemical stimulator(s) including RS-1, IP6 (Inositol Hexakisphosphate), DNA-PK enhancer and combinations thereof or chemical inhibitor(s) including Mirin and derivatives, inhibitors of PolQ, inhibitors of CtIP, RI-1, BO2 and combinations thereof.

38. A cell or a population of cells, comprising:
i) conditioned cell or population of cells,
ii) DNA double-strand breaks and/or DNA single-strand breaks in the genomic nucleic acid, and/or
iii) a modulation of one or more DNA Repair Pathways (DRPs), and wherein the genomic nucleic acid(s), of the cell or cells of the population of cells, comprise(s) the at least one alteration, preferably an insertion.
